(19) United States
(12) Patent Application Publication
(10) Pub. No.: US 2009/0230857 A1
(43) Pub. Date: Sep. 17, 2009

(54) AROMATIC HETEROCYCLIC COMPOUND, ORGANIC LIGHT EMITTING DIODE COMPRISING ORGANIC LAYER COMPRISING THE SAME AND METHOD OF MANUFACTURING THE ORGANIC LIGHT EMITTING DIODE

(76) Inventors: Byoung-ki Choi, Hwaseong-si (KR); O-hyun Kwon, Seoul (KR); Myeong-suk Kim, Hwaseong-si (KR); Dong-woo Shin, Seoul (KR); Yu-ri Choi, Yongin-si (KR); Duk-keun An, Chuncheon-si (KR); Seong-jib Choi, Chuncheon-si (KR)

Correspondence Address: F. CHAU & ASSOCIATES, LLC, 130 WOODBURY ROAD, WOODBURY, NY, 11797, US

(21) Appl. No.: 12/403,805

(22) Filed: Mar. 13, 2009

(30) Foreign Application Priority Data
Mar. 14, 2008 (KR) 10-2008-0024097
Sep. 12, 2008 (KR) 10-2008-0090500

(51) Int. Cl. H01J 1/63; C07D 471/04; C07D 471/22; B05D 5/00

(52) U.S. Cl. 313/504; 546/85; 546/82; 427/64

(57) ABSTRACT

Provided are an aromatic heterocyclic compound represented by Formula 1 below, an organic light emitting diode including the same, and a method of manufacturing the organic light emitting diode:

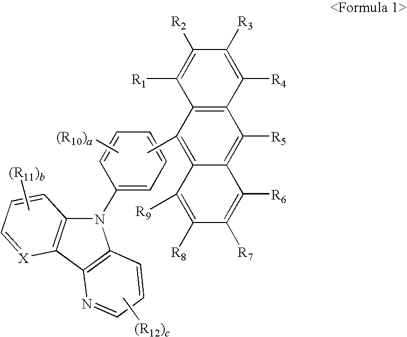

wherein descriptions of Formula 1 are the same as defined in the detailed description of the invention.

### CROSS-REFERENCE TO RELATED PATENT APPLICATION

This application claims the benefit of Korean Patent Application No. 10-2008-0024097, filed on Mar. 14, 2008 and Korean Patent Application No. 10-2008-0090500, filed on Sep. 12, 2008, the disclosures of which are each hereby incorporated by reference herein in their entireties.

### BACKGROUND OF THE INVENTION

1. Technical Field

The present disclosure relates to an aromatic heterocyclic compound, an organic light emitting diode including an organic layer comprising the aromatic heterocyclic compound, and to a method of manufacturing the organic light emitting diode. More particularly, the present disclosure relates to an aromatic heterocyclic compound that has beneficial light emitting properties, and is used to manufacture an organic light emitting diode which can operate at a low voltage, and have high efficiency, high luminance, high color purity and long lifetime, an organic light emitting diode including an organic layer comprising the aromatic heterocyclic compound, and to a method of manufacturing the organic light emitting diode.

2. Description of the Related Art

Organic light emitting diodes (OLEDS) may have good luminance, operating voltage and response time and can realize multicolor images. In this regard, much research has been conducted on OLEDs.

In general, OLEDs have a stacked structure of an anode, an organic emissive layer, and a cathode. OLEDs may also have various structures such as, for example, anode/hole injection layer/hole transport layer/emissive layer/electron transport layer/electron injection layer/cathode, anode/hole injection layer/hole transport layer/emissive layer/hole blocking layer/electron transport layer/electron injection layer/cathode, or the like.

The materials used in OLEDs can be classified into, for example, either vacuum deposition materials or solution coating materials according to a method of forming an organic layer. When manufacturing an OLED using a vacuum deposition process, the use of a vacuum system and a shadow mask to form pixels for a natural color display may be required. On the other hand, in the case of manufacturing an OLED using a solution coating process such as, for example, inkjet printing, screen printing, or spin coating, it may be relatively easy to prepare an organic layer, manufacturing costs may be low, and a relatively good resolution can be achieved as compared to the case of using a shadow mask.

Therefore, there is still a need to develop a compound having high thermal stability and light-emitting properties, and which can be used to form an organic layer of an organic light emitting diode, regardless of the method used for forming the organic layer.

### SUMMARY OF THE INVENTION

In accordance with an exemplary embodiment of the present invention, an aromatic heterocyclic compound represented by Formula 1 as set forth below is provided:

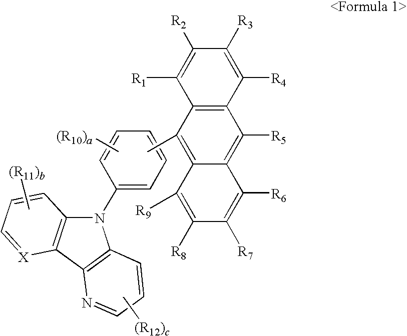

wherein R₁ through R₉ are each independently hydrogen, halogen, a cyano group, a nitro group, a hydroxyl group, a substituted or unsubstituted C₁-C₂₀ alkyl group, a substituted or unsubstituted C₂-C₂₀ alkenyl group, a substituted or unsubstituted C₂-C₂₀ alkynyl group, a substituted or unsubstituted C₅-C₂₀ cycloalkyl group, a substituted or unsubstituted C₅-C₂₀ cycloalkenyl group, a substituted or unsubstituted C₅-C₂₀ aryl group, a substituted or unsubstituted C₂-C₃₀ heteroaryl group, or a group represented by —N(Z₁)(Z₂) where Z₁ and Z₂ are each independently hydrogen, a substituted or unsubstituted C₁-C₂₀ alkyl group, or a substituted or unsubstituted C₅-C₂₀ aryl group,

R₁₀ through R₁₂ are each independently hydrogen, halogen, a cyano group, a nitro group, a hydroxyl group, a substituted or unsubstituted C₁-C₂₀ alkyl group, a substituted or unsubstituted C₂-C₂₀ alkenyl group, a substituted or unsubstituted C₂-C₂₀ alkynyl group, a substituted or unsubstituted C₅-C₂₀ cycloalkyl group, a substituted or unsubstituted C₅-C₂₀ cycloalkenyl group, a substituted or unsubstituted C₅-C₂₀ aryl group, a substituted or unsubstituted C₂-C₂₀ heteroaryl group, a group represented by —N(Z₁)(Z₂) where Z₁ and Z₂ are each independently hydrogen, a substituted or unsubstituted C₁-C₂₀ alkyl group, or a substituted or unsubstituted C₅-C₂₀ aryl group, or a group represented by -Q₁-Q₂ where Q₁ is a C₅-C₂₀ arylene group or a C₂-C₂₀ heteroarylene group, and Q₂ is a substituted or unsubstituted C₅-C₂₀ aryl group or a substituted or unsubstituted C₂-C₂₀ heteroaryl group,

a, b, and c are each independently 0, 1 or 2; and

X is CY₁ or N, wherein Y₁ is hydrogen, a substituted or unsubstituted C₁-C₂₀ alkyl group, or a substituted or unsubstituted C₁-C₂₀ alkoxy group.

In accordance with an exemplary embodiment of the present invention, an organic light emitting diode is provided. The organic light emitting diode comprising a first electrode, a second electrode, and an organic layer comprising the aromatic heterocyclic compound described above between the first electrode and the second electrode.

In accordance with an exemplary embodiment of the present invention, a method of manufacturing an organic light emitting diode is provided. The method comprising forming a first electrode on a substrate, forming an organic layer comprising the aromatic heterocyclic compound described above on the first electrode and forming a second electrode on the organic layer.

### BRIEF DESCRIPTION OF THE DRAWINGS

Exemplary embodiments of the present invention can be understood in more detail from the following description taken in conjunction with the attached drawings in which:

FIGS. 1A through 1C are schematic cross-sectional views respectively illustrating structures of organic light emitting diodes according to an exemplary embodiment of the present invention;

FIG. 2 is a graph showing MS chromatography of Compound 1 according to an exemplary embodiment of the present invention;

FIGS. 3A and 3B are graphs showing differential scanning calorimetry (DSC) data of Compound 1 according to an exemplary embodiment of the present invention;

FIG. 4 is a graph showing thermogravimetric analysis (TGA) data of Compound 1 according to an exemplary embodiment of the present invention;

FIG. 5 is a graph showing a UV absorption spectrum and a photoluminescence (PL) spectrum of Compound 1 according to an exemplary embodiment of the present invention;

FIG. 6 shows graphs of MS chromatography of Compound 6 according to an exemplary embodiment of the present invention;

FIGS. 7A and 7B are graphs showing DSC data of Compound 6 according to an exemplary embodiment of the present invention;

FIG. 8 is a graph showing TGA data of Compound 6 according to an exemplary embodiment of the present invention;

FIG. 9 shows graphs of MS chromatography of Compound 7 according to an exemplary embodiment of the present invention;

FIGS. 10A and 10B are graphs showing DSC data of Compound 7 according to an exemplary embodiment of the present invention;

FIG. 11 is a graph showing TGA data of Compound 7 according to an exemplary embodiment of the present invention; and

FIG. 12 is a graph showing brightness of each of Samples 1, 2, 3, 4, and A (Examples 1 through 4 and Comparative Example 1);

FIG. 13 is a graph showing current density of each of Samples 1, 2, 3, 4, and A (Examples 1 through 4 and Comparative Example 1);

FIG. 14 is a graph showing efficiency of each of Samples 1, 2, 3, 4, and A (Examples 1 through 4 and Comparative Example 1); and

FIG. 15 is a graph showing power efficiency of each of Samples 1, 2, 3, 4, and A (Examples 1 through 4 and Comparative Example 1).

### DETAILED DESCRIPTION OF THE EXEMPLARY EMBODIMENTS OF THE INVENTION

The present invention will now be described more fully with reference to the accompanying drawings, in which exemplary embodiments of the invention are shown.

Exemplary embodiments of the present invention provide an aromatic heterocyclic compound represented by Formula 1 below:

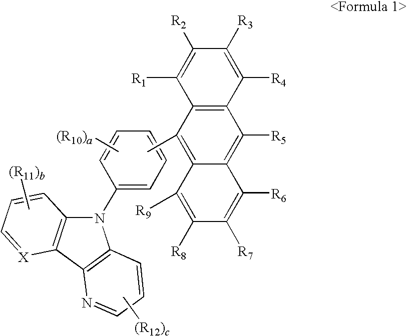

wherein R₁₀ through R₁₂ may be, for example, each independently hydrogen, halogen, a cyano group, a nitro group, a hydroxyl group, a substituted or unsubstituted C₁-C₂₀ alkyl group, a substituted or unsubstituted C₂-C₂₀ alkenyl group, a substituted or unsubstituted C₂-C₂₀ alkynyl group, a substituted or unsubstituted C₅-C₂₀ cycloalkyl group, a substituted or unsubstituted C₅-C₂₀ cycloalkenyl group, a substituted or unsubstituted C₅-C₂₀ aryl group, a substituted or unsubstituted C₂-C₂₀ heteroaryl group, a group represented by —N(Z₁)(Z₂) where Z₁ and Z₂ are each independently hydrogen, a substituted or unsubstituted C₁-C₂₀ alkyl group, or a substituted or unsubstituted C₅-C₂₀ aryl group, or a group represented by -Q₁-Q₂ where Q₁ is a C₅-C₂₀ arylene group or a C₂-C₂₀ heteroarylene group, and Q₂ is a substituted or unsubstituted C₅-C₂₀ aryl group or a substituted or unsubstituted C₂-C₂₀ heteroaryl group.

R₁₀ through R₁₂ as described above may each independently exist in the position as indicated in Formula 1A below:

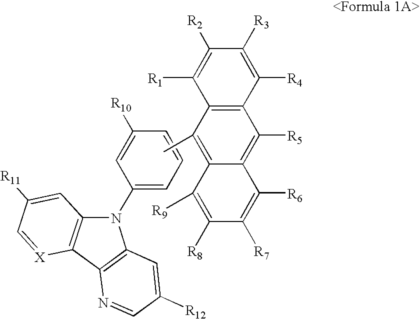

For example, R₁₀ through R₁₂ may be each independently hydrogen, a substituted or unsubstituted C₁-C₂₀ alkyl group, a substituted or unsubstituted C₅-C₂₀ aryl group, a substituted or unsubstituted C₂-C₂₀ heteroaryl group, or a group represented by -Q₁-Q₂ where Q₁ is a C₅-C₂₀arylene group, and Q₂ is a substituted or unsubstituted C₅-C₂₀ aryl group, or a substituted or unsubstituted C₂-C₂₀ heteroaryl group, and more particularly, R₁₀ through R₁₂ may be, for example, each independently hydrogen, a substituted or unsubstituted C₁-C₁₀ alkyl group, a substituted or unsubstituted C₅-C₁₄ aryl group, a substituted or unsubstituted C₂-C₁₄ heteroaryl group, or a group represented by -Q₁-Q₂ where Q₁ is a C₅-C₁₄ arylene group, and Q₂ is a substituted or unsubstituted C₅-C₁₄ aryl group, or a substituted or unsubstituted C₂-C₁₄heteroaryl group.

Substituents of R₁₀ through R₁₂ as described above may include, for example, a halogen atom, a hydroxyl group, a nitro group, a cyano group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, a C₁-C₁₀ alkoxy group, a C₆-C₂₀ aryl group, and a C₂-C₂₀ heteroaryl group, but the exemplary embodiments of the present invention are not limited thereto. For example, substituents of R₁₀ through R₁₂ may include a C₁-C₁₀ alkyl group and a C₆-C₂₀ aryl group.

In Formula 1, a, b, and c may be, for example, each independently 0, 1, or 2.

For example, R₁₀ through R₁₂ of Formula 1 may be each independently hydrogen or one of the structures represented by Formula 2a to 2o below, but the exemplary embodiments of the present invention are not limited thereto:

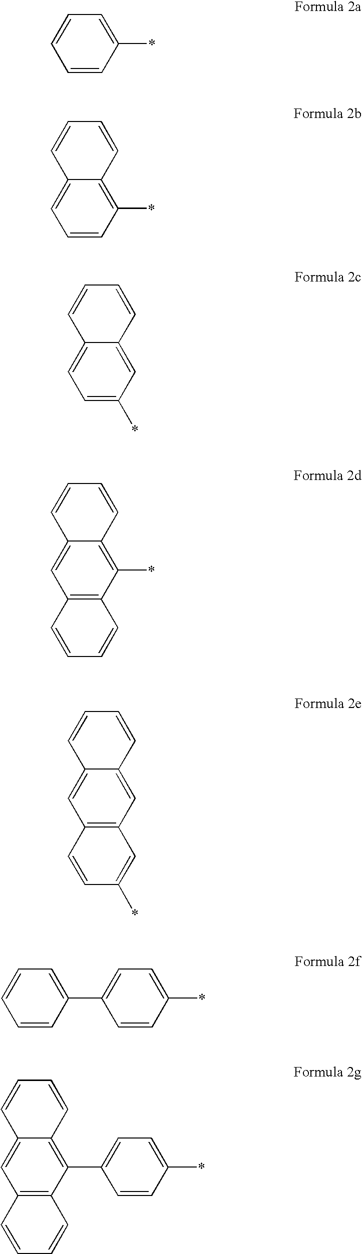

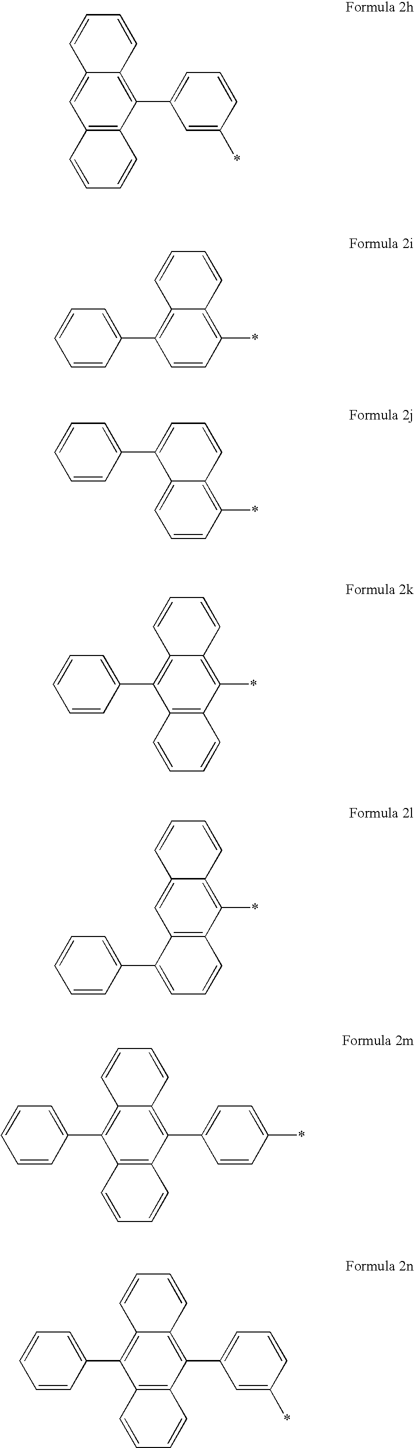

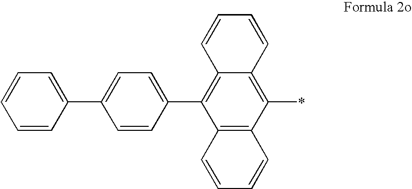

In Formula 1, X is CY₁ or N, wherein Y₁ is hydrogen, a substituted or unsubstituted C₁-C₂₀ alkyl group or a substituted or unsubstituted C₁-C₂₀ alkoxy group.

Substituents of Y₁ as described above may include, for example, a halogen atom, a hydroxyl group, a nitro group, a cyano group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, a C₁-C₁₀ alkoxy group, a C₆-C₂₀ aryl group, and a C₂-C₂₀ heteroaryl group, but the present invention is not limited thereto. For example, substituents of Y₁ may include a C₁-C₁₀ alkyl group or a C₆-C₂₀ aryl group.

For example, Y₁ may be a substituted or unsubstituted C₁-C₁₀ alkyl group or a substituted or unsubstituted C₁-C₁₀ alkoxy group.

According to an exemplary embodiment of the present invention, an aromatic heterocyclic compound may be represented by Formula 1 where X is CH or N, but the exemplary embodiments of the present invention are not limited thereto.

In Formula 1, R₁ through R₉ may be, for example, each independently hydrogen, halogen, a cyano group, a nitro group, a hydroxyl group, a substituted or unsubstituted C₁-C₂₀ alkyl group, a substituted or unsubstituted C₂-C₂₀ alkenyl group, a substituted or unsubstituted C₂-C₂₀ alkynyl group, a substituted or unsubstituted C₅-C₂₀ cycloalkyl group, a substituted or unsubstituted C₅-C₂₀ cycloalkenyl group, a substituted or unsubstituted C₅-C₂₀ aryl group, a substituted or unsubstituted C₂-C₃₀ heteroaryl group, or a group represented by —N(Z₁)(Z₂) where Z₁ and Z₂ may be each independently hydrogen, a substituted or unsubstituted C₁-C₂₀ alkyl group, or a substituted or unsubstituted C₅-C₂₀ aryl group.

Substituents of R₁ through R₉ as described above may include, for example, a halogen atom, a hydroxyl group, a nitro group, a cyano group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, a C₁-C₁₀ alkoxy group, a C₆-C₂₀ aryl group, and a C₂-C₂₀ heteroaryl group, but the present invention is not limited thereto. For example, substituents of R₁ through R₉ may include a C₁-C₁₀ alkyl group or a C₆-C₂₀ aryl group.

For example, R₁ through R₉ may be each independently hydrogen, a substituted or unsubstituted C₅-C₂₀ aryl group, or a substituted or unsubstituted C₂-C₃₀ heteroaryl group. For example, when R₁ through R₉ are each independently hydrogen, a substituted or unsubstituted C₅-C₂₀ aryl group, or a substituted or unsubstituted C₂-C₃₀ heteroaryl group, an organic layer comprising the aromatic heterocyclic compound of Formula 1 can have improved film-forming ability and increased stability. For example, R₁ through R₉ may be each independently hydrogen, a substituted or unsubstituted C₅-C₁₄ aryl group, or a substituted or unsubstituted C₂-C₁₄ heteroaryl group.

For example, R₁ through R₄ and R₆ through R₉ are hydrogen, R₅ is hydrogen, a substituted or unsubstituted C₅-C₂₀ aryl group, or a substituted or unsubstituted C₂-C₃₀ heteroaryl group, but the present invention is not limited thereto.

For example, R₁ through R₉ may be each independently hydrogen or one of the structures represented by Formula 3a to 3i below, but the exemplary embodiments of the present invention are not limited thereto:

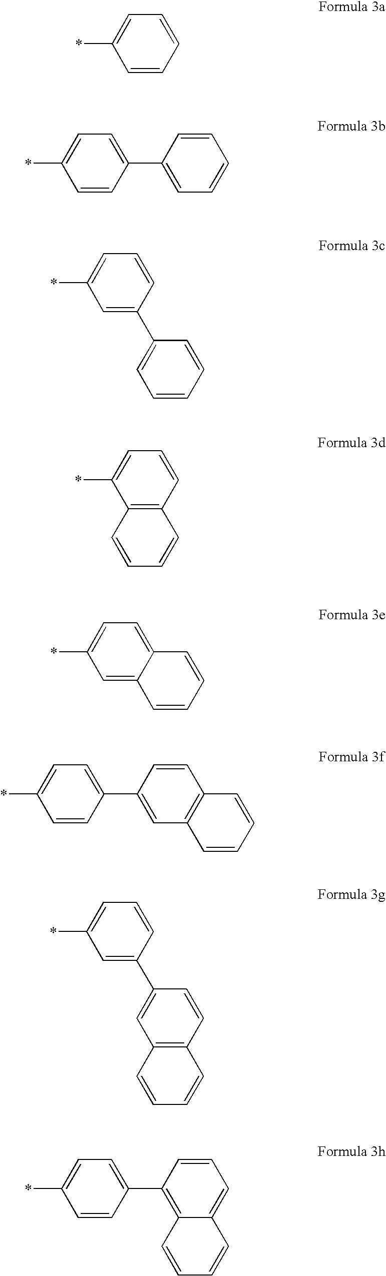

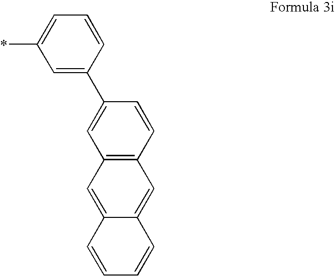

For example, the aromatic heterocyclic compound according to exemplary embodiments of the present invention may be represented by Formula 1B below:

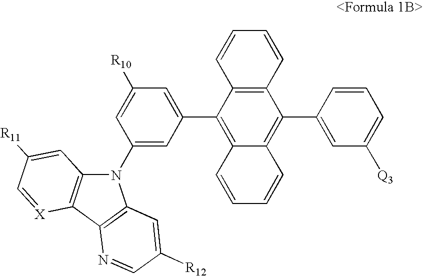

wherein R₁₀ through R₁₂ and X are the same as defined above.

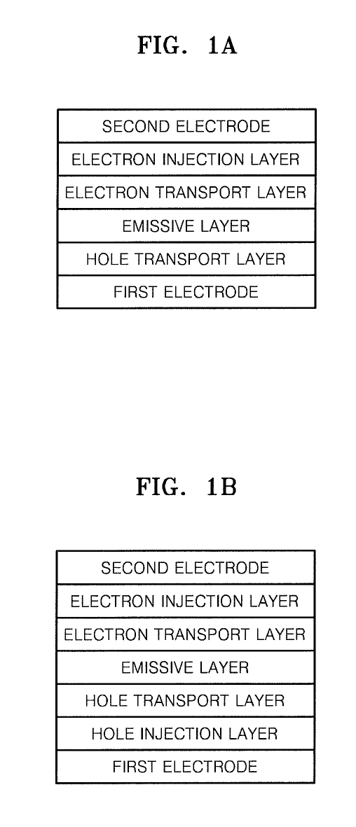

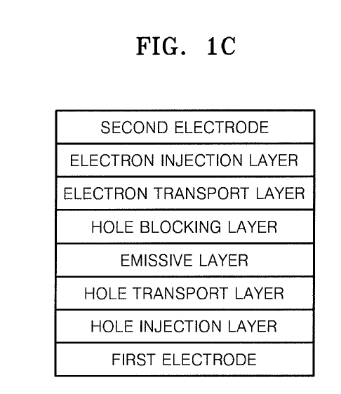

In FIG. 1B, Q₃ is hydrogen or an unsubstituted C₅-C₂₀ aryl group, and preferably hydrogen or an unsubstituted C₅-C₁₄ aryl group.

Examples of the unsubstituted C₁-C₂₀ alkyl group used herein include but are not limited to methyl, ethyl, propyl, isobutyl, sec-butyl, pentyl, iso-amyl, hexyl, and the like. At least one hydrogen atom of the alkyl group described above can be substituted with, for example, a halogen atom, a hydroxyl group, a nitro group, a cyano group, an amino group, an amidino group, hydrazine, hydrazone, a carboxyl group or salts thereof, a sulfonic acid group or salts thereof, a phosphoric acid or salts thereof, a C₁-C₃₀ alkyl group, a C₁-C₃₀ alkoxy group, a C₁-C₃₀ alkenyl group, a C₁-C₃₀ alkynyl group, a C₆-C₃₀ aryl group, or a C₂-C₂₀ heteroaryl group.

Examples of the unsubstituted C₁-C₂₀ alkoxy group used herein include but are not limited to methoxy, ethoxy, propoxy, and the like. At least one hydrogen atom of the alkoxy group can be substituted with, for example, the same substituents as those of the substituted C₁-C₂₀ alkyl group described above.

The unsubstituted C₂-C₂₀ alkenyl group used herein contains a carbon-carbon double bond in the middle or end of the alkyl group defined above. Examples of the unsubstituted C₂-C₂₀ alkenyl group include but are not limited to ethylene, propylene, butylene, hexylene and the like. At least one hydrogen atom of the alkenyl groups can be substituted with, for example, the same substituents as those of the substituted C₁-C₂₀ alkyl group described above.

The unsubstituted C₂-C₂₀ alkynyl group used herein contains a carbon-carbon triple bond in the middle or end of the alkyl group defined above. At least one hydrogen atom of the alkynyl groups can be substituted with, for example, the same substituents as those of the substituted C₁-C₂₀ alkyl group described above. Examples of the unsubstituted C₂-C₂₀ alkynyl group include but are not limited to acetylene, propylene, phenylacetylene, naphthylacetylene, isopropylacetylene, t-butylacetylene, diphenylacetylene, and the like.

The unsubstituted C₅-C₂₀ aryl group refers to a C₅-C₂₀ carbocyclic aromatic system containing at least one aromatic ring. At least two aromatic rings can be fused with each other, or bound to each other by a single bond, or the like. At least one hydrogen atom of the aryl groups can be substituted with, for example, the same substituents as those of the substituted C₁-C₂₀ alkyl group described above.

The substituted or unsubstituted C₅-C₂₀ aryl group can be, for example, a phenyl group, a C₁-C₁₀ alkylphenyl group (for example, an ethylphenyl group), a halophenyl group (for example, o-, m- and p-fluorophenyl groups and a dichlorophenyl group), a cyanophenyl group, a dicyanophenyl group, a trifluoro methoxy phenyl group, a biphenyl group, a halobiphenyl group, a cyanobiphenyl group, a C₁-C₁₀ biphenyl group, a C₁-C₁₀ alkoxybiphenyl group, o-, m-, and p-tolyl groups, o-, m- and p-cumenyl group, a mesityl group, a phenoxy phenyl group, a (α,α-dimethylbenzene)phenyl group, a (N,N′-dimethyl)aminophenyl group, a (N,N′-diphenyl)aminophenyl group, a pentalenyl group, an indenyl group, a naphthyl group, a halonaphthyl group (for example, a fluoronaphthyl group), a C₁-C₁₀ alkylnaphthyl group (for example, a methylnaphthyl group), a C₁-C₁₀ alkoxynaphthyl group (for example, a methoxynaphthyl group), a cyanonaphthyl group, an anthracenyl group, an azulenyl group, or the like. These aryl group can be substituted with, for example, the same substituents as those of the substituted C₁-C₂₀ alkyl group described above.

The unsubstituted C₅-C₂₀ arylene group used herein is a bivalent linker having a structure similar to that of the unsubstituted C₅-C₂₀ aryl group, and examples thereof include a phenylene group, a naphthylene group, and the like. However, exemplary embodiments of the present invention are not limited thereto. At least one hydrogen atom of the arylene group can be substituted with, for example, the same substituents as those of the substituted C₁-C₂₀ alkyl group described above.

The unsubstituted C₂-C₂₀ heteroaryl group refers to a system comprising at least one aromatic ring that includes at least one hetero atom selected from N, O, P, and S and the remaining ring member is carbon. The one or more aromatic rings can be fused with each other, or bound to each other by a single bond, or the like. At least one hydrogen atom of the heteroaryl groups can be substituted with, for example, the same substituents as those of the substituted C₁-C₂₀ alkyl group described above.

Examples of the unsubstituted C₂-C₂₀ heteroaryl group may include but are not limited to a pyrazolyl group, an imidazolyl group, an oxazolyl group, a thiazolyl group, a triazolyl group, a tetrazolyl group, an oxadiazolyl group, a pyridinyl group, a pyridazinyl group, a pyrimidinyl group, a triazinyl group, a carbazolyl group, an indolyl group, a quinolinyl group, an isoquinolinyl group, and the like. These heteroaryl groups can be substituted with, for example, the same substituents as those of the substituted C₁-C₂₀ alkyl group described above.

The unsubstituted C₅-C₂₀ cycloalkyl group refers to an alkyl group having a ring system, and the unsubstituted C₅-C₂₀ cycloalkenyl group refers to an alkenyl group having a ring system. At least one hydrogen atom of the cycloalkyl group and the cycloalkenyl group can be each independently substituted with, for example, the same substituents as those of the substituted C₁-C₂₀ alkyl group described above.

According to an exemplary embodiment of the present invention, the aromatic heterocyclic compound of Formula 1 may be one of Compounds 1 through 17 below, but the exemplary embodiments of the present invention are not limited thereto:

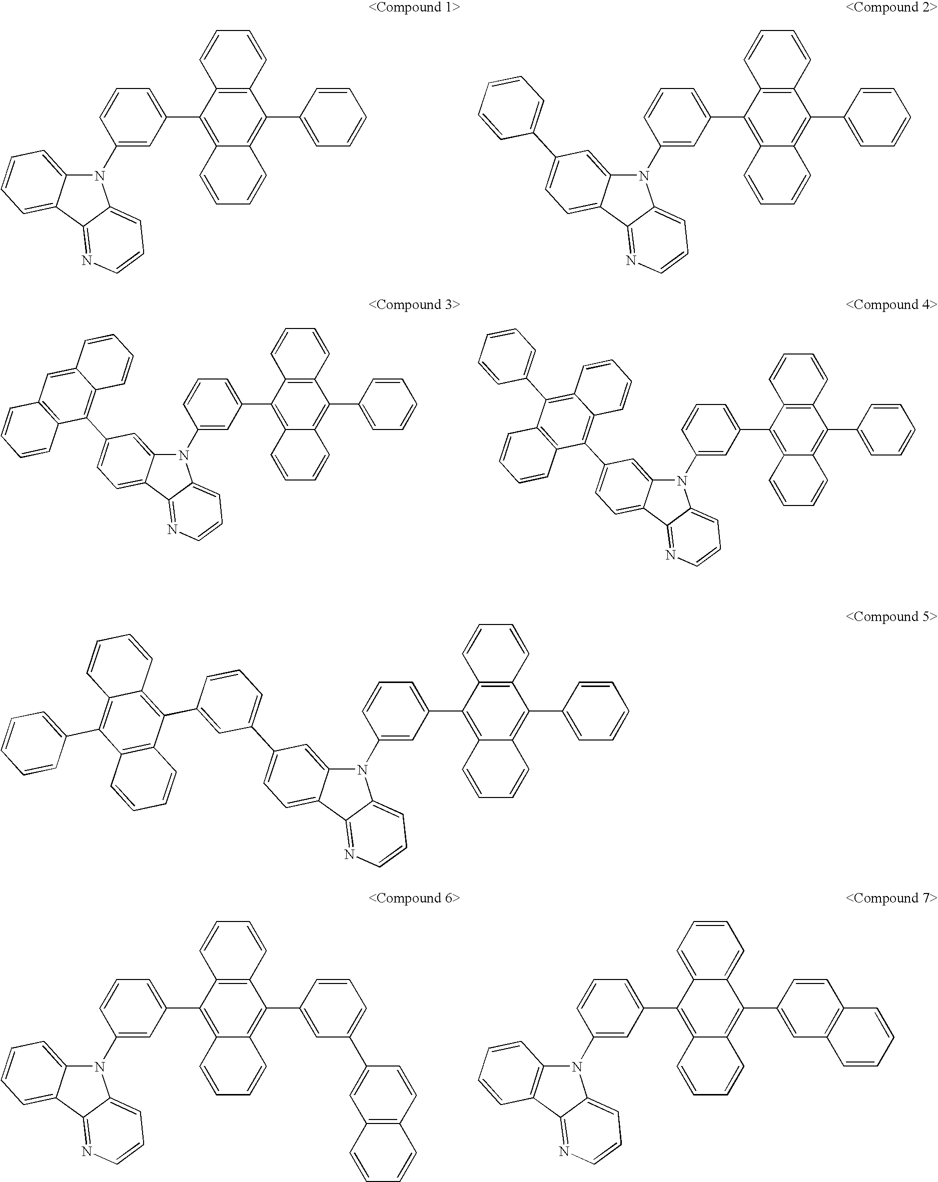

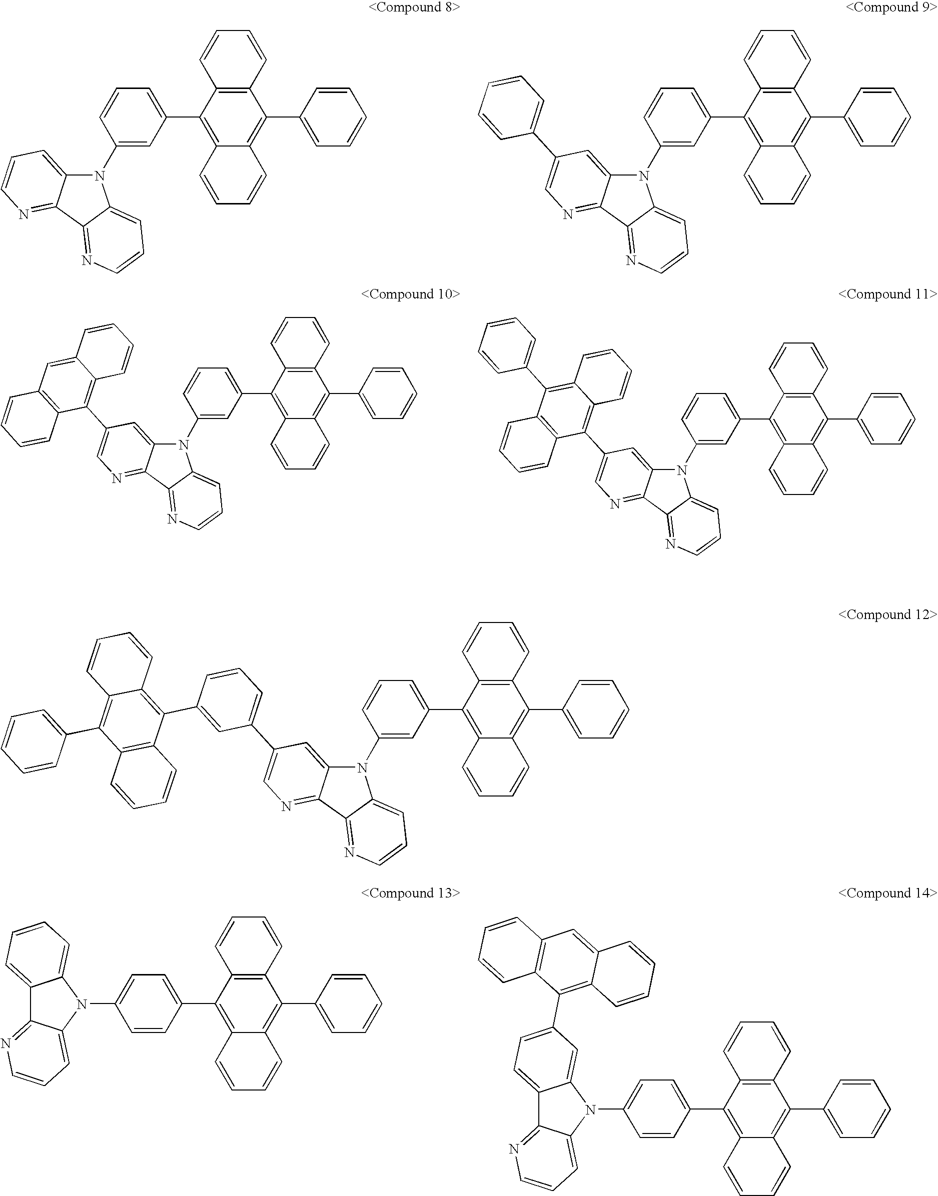

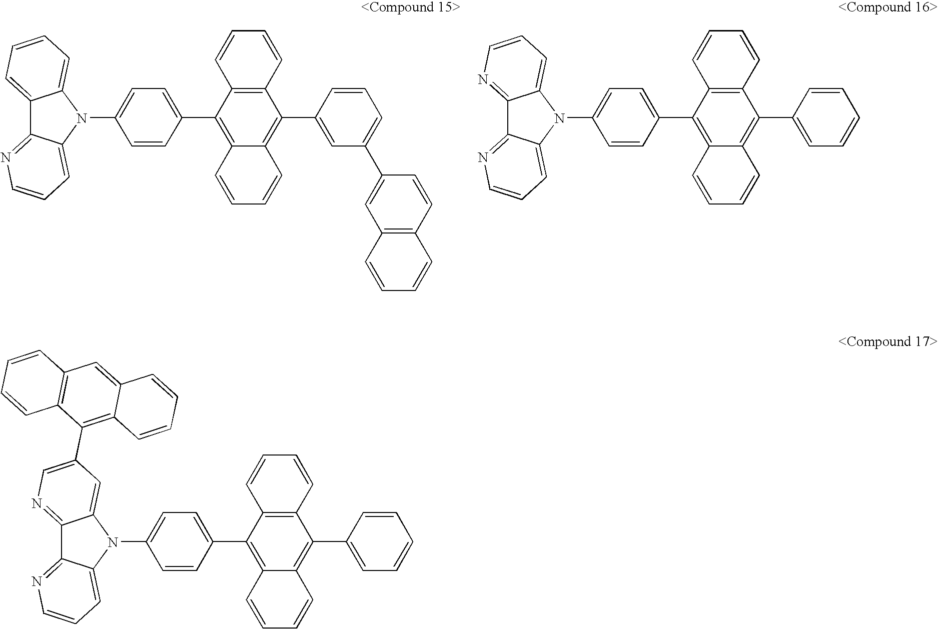

The aromatic heterocyclic compound of Formula 1 may be synthesized using, for example, organic synthesis.

The aromatic heterocyclic compound of Formula 1 as described above may be included in an organic layer of an organic light emitting diode. Thus, exemplary embodiments of the present invention also provide an organic light emitting diode including a first electrode, a second electrode, and an organic layer that is disposed between the first electrode and the second electrode, and includes the aromatic heterocyclic compound of Formula 1 as described above.

For example, the organic layer may be an emissive layer or an electron transporting layer.

The organic layer comprising the aromatic heterocyclic compound of Formula 1 may be formed using various known methods. For example, the organic layer may be formed by vacuum deposition or solution coating, such as spin coating, inkjet printing, screen printing, casting, Langmuir Blodgett (LB), spray printing, or the like. In addition, the organic layer comprising the aromatic heterocyclic compound of Formula 1 may be formed, for example, on a donor film using vacuum deposition or solution coating as described above, and then thermally transferred to a substrate on which a first electrode, and the like are formed. The aromatic heterocyclic compound of Formula 1 has beneficial solubility and thermal stability and can also be used to form a stable organic layer. Thus, an organic light emitting diode which can operate at low voltage, and have high efficiency and high luminance can be obtained.

The organic light emitting diode of exemplary embodiments of the present invention may further include, between the first electrode and the second electrode, at least one layer selected from the group consisting of a hole injection layer, a hole transport layer, an emissive layer, a hole blocking layer, an electron transport layer, and an electron injection layer. For example, the structures of organic light emitting diodes according to embodiments of the present invention are illustrated in FIGS. 1A, 1B and 1C. The organic light emitting diode of FIG. 1A has a structure comprising a first electrode, a hole transport layer, an emissive layer, an electron transport layer, an electron injection layer, and a second electrode. The organic light emitting diode of FIG. 1B has a structure comprising a first electrode, a hole injection layer, a hole transport layer, an emissive layer, an electron transport layer, an electron injection layer, and a second electrode. In addition, the organic light emitting diode of FIG. 1C has a structure comprising a first electrode, a hole injection layer, a hole transport layer, an emissive layer, a hole blocking layer, an electron transport layer, an electron injection layer, and a second electrode. Herein, at least one of the emissive layer, the hole injection layer, the hole transport layer, the hole blocking layer, and the electron transport layer may comprise the aromatic heterocyclic compound of Formula 1.

Hereinafter, a method of manufacturing an organic light emitting diode, according an exemplary embodiment of the present invention, will be described with reference to the organic light emitting diode illustrated in FIG. 1C.

First, a first electrode is formed by, for example, depositing or sputtering a high work-function material for a first electrode on a substrate. The first electrode can be an anode. The substrate, which can be any substrate that is used in conventional organic light emitting diodes, may be, for example, a glass substrate or a transparent plastic substrate that has excellent mechanical strength, thermal stability, transparency, and surface smoothness, can be easily treated, and is waterproof. The first electrode can be formed of, for example, ITO, indium zinc IZO, SnO₂, ZnO, or any transparent material which has high conductivity.

Next, a hole injection layer (HIL) may be formed on the first electrode by, for example, vacuum deposition, spin coating, casting, Langmuir Blodgett (LB) deposition, or the like.

When the HIL is formed by vacuum deposition, vacuum deposition conditions may vary according to the compound that is used to form the HIL, and the desired structure and thermal properties of the HIL to be formed. In general, however, the vacuum deposition may be performed, for example, at a deposition temperature of about 100 to about 500° C., a pressure of about 10⁻⁸ to about 10⁻³ torr, and a deposition speed of about 0.01 to about 100 Å/sec.

When the HIL is formed by spin coating, coating conditions may vary according to the compound that is used to form the HIL, and the desired structure and thermal properties of the HIL to be formed. In general, however, the coating speed may be in a range of, for example, about 2,000 to about 5,000 rpm, and a temperature for heat treatment, which is performed to remove a solvent after coating, may be in a range of, for example, about 80 to about 200° C.

The HIL can be formed of any known materials used to form a HIL. For example, the material may m-MTDATA 4,4′,4″-tris(3-methylphenylphenylamino)triphenylamine), NPB (N,N′-di(1-naphthyl)-N,N′-diphenyl benzidine), TDATA, 2T-NATA, polyaniline/Dodecylbenzenesulfonic acid (Pani/DBSA); poly(3,4-ethylenedioxythiophene)/poly(4-styrenesulfonate) (PEDOT/PSS); polyaniline/camphor sulfonic acid (Pani/CSA); (polyaniline)/poly(4-styrenesulfonate) (PANI/PSS); or the like.

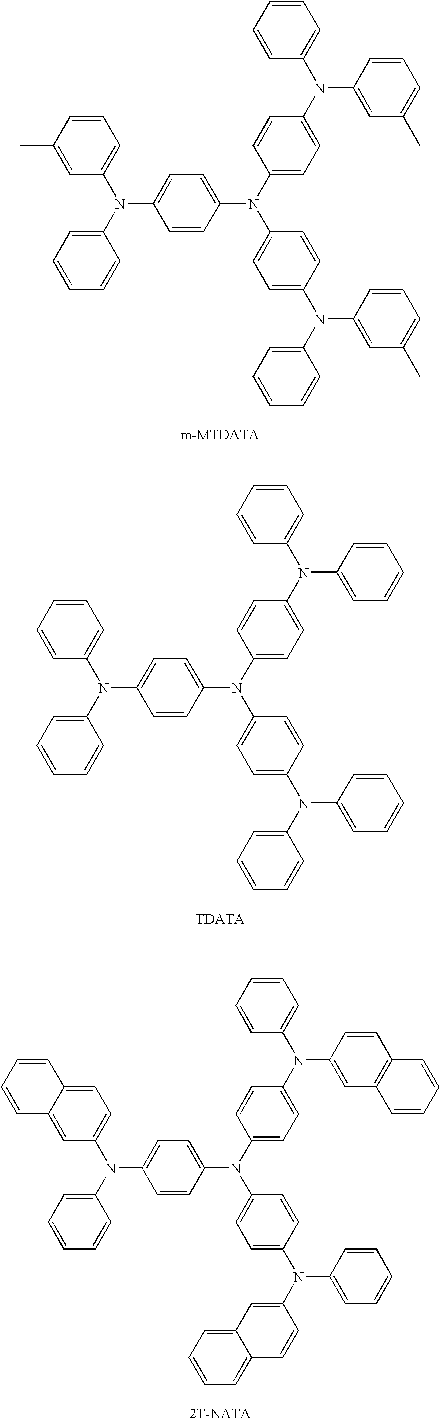

The thickness of the HIL may be in the range of, for example, about 100 to about 10,000 Å, and particularly, in the range of about 100 to about 1,000 Å. When the thickness of the HIL is within the ranges described above, a satisfactory hole injecting ability of the HIL can be obtained without a substantial decrease in a driving voltage of the organic light emitting diode.

Next, a hole transport layer (HTL) may be formed on the HIL by, for example, vacuum deposition, spin coating, casting, LB deposition, or the like. When the HTL is formed by vacuum deposition or spin coating, the conditions for deposition and coating are similar to those for the formation of the HIL, although conditions for the deposition and coating may vary according to the compound that is used to form the HTL.

The HTL may be formed of any known materials used to form a HTL. For example, the HTL may be formed of a carbazole derivative, such as N-phenylcarbazole, polyvinylcarbazole, or the like; a typical amine derivative having an aromatic condensation ring such as N,N′-bis(3-methylphenyl)-N,N′-diphenyl-[1,1-biphenyl]-4,4′-diamine(TPD), N,N′-di(naphthalene-1-yl)-N,N′-diphenyl benzydine (α-NPD), triphenylamin-based material such as TCTA (4,4′,4″-tris(N-carbazolyl)triphenylamine); or the like.

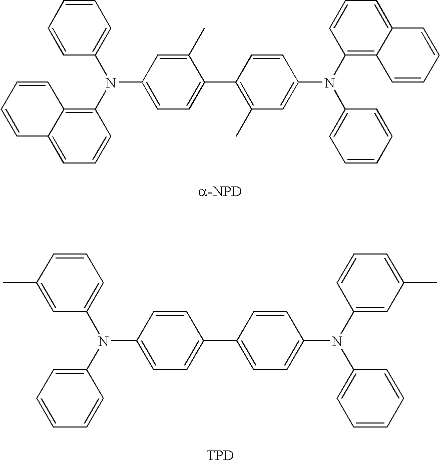

The thickness of the HTL may be in a range of, for example, about 50 to about 1,000 Å, and particularly, in a range of about 100 to about 800 Å. When the thickness of the HTL is within the ranges described above, a satisfactory hole transporting ability of the HTL can be obtained without a substantial decrease in a driving voltage of the organic light emitting diode.

Next, an emissive layer (EML) may be formed on the HTL by, for example, vacuum deposition, spin coating, casting, LB deposition, or the like. When the EML is formed by vacuum deposition or spin coating, the conditions for deposition and coating are similar to those for the formation of the HIL, although the conditions for deposition and coating may vary according to the compound that is used to form the EML.

The EML may comprise the aromatic heterocyclic compound of Formula 1 according to the present invention, as described above. Herein, the aromatic heterocyclic compound of Formula 1 may be used, as a dopant, together with a know host material, and the EML may further comprise a known dopant material. In addition, the aromatic heterocyclic compound of Formula 1 may be used as a host. Alternatively, the aromatic heterocyclic compound of Formula 1 may be used alone. The host material may be, for example, Alq₃, 4,4′-N,N′-dicarbazole-biphenyl (CBP), poly(n-vinylcarbazole) (PVK), 9,10-di(naphthyl)anthracene (AND), or the like, but the present invention is not limited thereto.

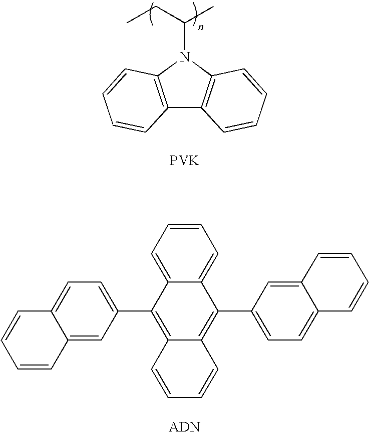

A known red dopant may be PtOEP, Ir(piq)₃, Btp₂Ir(acac), DCJTB, or the like, but the present invention is not limited thereto.

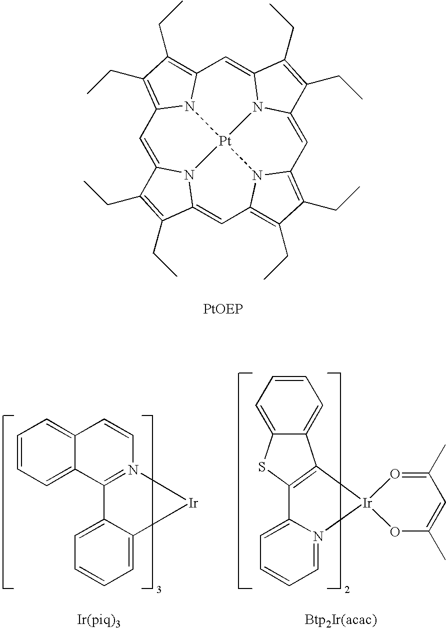

In addition, a known green dopant may be Ir(ppy)₃ (ppy=phenylpyridine), Ir(ppy)₂(acac), Ir(mpyp)₃, C545T, or the like, but the present invention is not limited thereto.

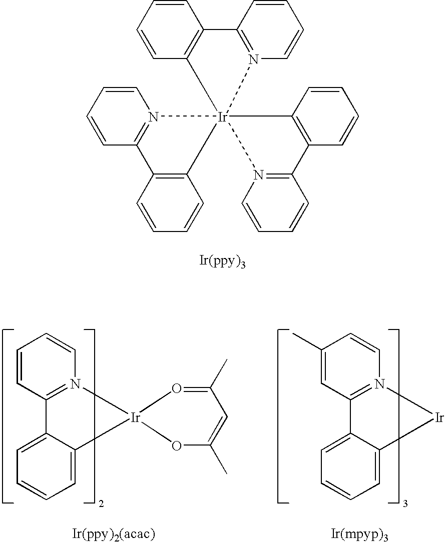

A known blue dopant may be F₂Irpic, (F₂ppy)₂Ir(tmd), Ir(dfppz)₃, ter-fluorene, 4,4′-bis(4-diphenylaminostyryl)biphenyl (DPAVBi), 2,5,8,11-tetra-tert-butylperylene (TBP), or the like, but the present invention is not limited thereto.

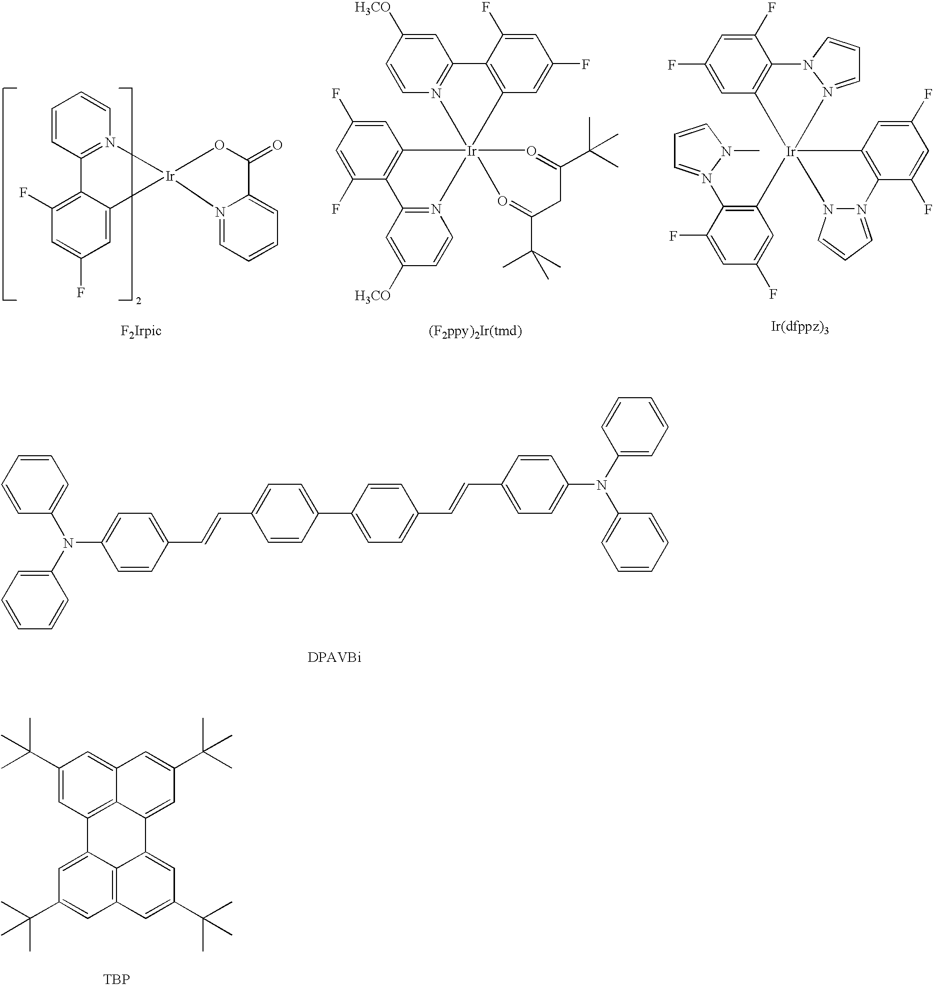

When the dopant and the host are used together, the doping concentration of the dopant is not particularly limited. In general, however, the amount of the dopant may be in a range of, for example, about 0.01 to about 15 parts by weight based on 100 parts by weight of the host.

The thickness of the EML may be in a range of, for example, about 100 to about 1,000 Å, and particularly, in a range of about 200to about 600 Å. When the thickness of the EML is within the ranges described above, the EML can have beneficial light emitting properties without a substantial decrease in a driving voltage of the organic light emitting diode.

When the EML includes a phosphorous dopant, to prevent triplet excitons or holes from migrating into an electron transport layer (ETL), a hole blocking layer (HBL) may be formed between the ETL and the EML by, for example, vacuum deposition, spin coating, casting, LB deposition, or the like. When the HBL is formed by vacuum deposition or spin coating, the conditions for deposition and coating are similar to those for the formation of the HIL, although the conditions for deposition and coating may vary according to the compound that is used to form the HBL. Here, a material used to form the HBL can be any known material used to form a HBL. Examples of such material may include but are not limited to oxadiazol derivatives, triazol derivatives, phenanthroline derivatives, and the like.

The thickness of the HBL may be in a range of, for example, about 50 to about 1,000 Å, and preferably, in a range of about 100 to about 300 Å. When the thickness of the HBL is within the ranges described above, the EML can have beneficial hole blocking properties without a substantial decrease in a driving voltage of the organic light emitting diode.

Next, an electron transport layer (ETL) is formed on the EML or HBL by, for example, vacuum deposition, spin coating, casting, or the like. When the ETL is formed by vacuum deposition or spin coating, the conditions for deposition and coating are, in general, similar to those for the formation of the HIL, although the conditions for deposition and coating may vary according to the material that is used to form the ETL. The material used to form the ETL should be able to stably transport electrons injected from an electron injecting electrode (cathode), and may be the aromatic heterocyclic compound of Formula 1 as described above. In addition, a known electron transporting material, such as, for example, a quinoline derivative, in particular, tris(8-quinolinorate)aluminum (Alq₃), TAZ, Balq, or the like can be used to form the ETL, but the exemplary embodiments of the present invention are not limited thereto.

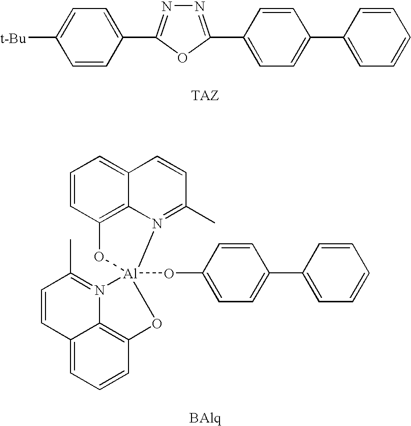

The thickness of the ETL may be in a range of, for example, about 100 to about 1,000 Å, and preferably, in a range of about 150 to about 500 Å. When the thickness of the ETL is within the ranges described above, satisfactory electron transporting properties of the ETL can be obtained without a substantial decrease in a driving voltage of the organic light emitting diode.

In addition, an electron injection layer (EIL) that makes it relatively easy for electrons to be injected from a cathode may be formed on the ETL, and a material used to form the EIL is not particularly limited.

The material used to form the EIL may be the aromatic heterocyclic compound of Formula 1 as described above. In addition, a known material, such as, for example, LiF, NaCl, CsF, Li₂O, BaO, or the like can be used as the material used to form the EIL. The conditions for deposition are, in general, similar to those for the formation of the HIL, although the conditions for deposition may vary according to the compound used to form the EIL.

The thickness of the EIL may be in a range of, for example, about 1 to about 100 Å, and particularly, in a range of about 5 to about 50 Å. When the thickness of the EIL is within the ranges described above, the EIL can have satisfactory electron injecting properties without a substantial decrease in a driving voltage of the organic light emitting diode.

Finally, a second electrode may be formed on the EIL by, for example, vacuum deposition, sputtering, or the like. The second electrode can be used as a cathode. The second electrode may be formed of, for example, a low work-function metal, an alloy, an electrically conductive compound, or a combination thereof. For example, the second electrode may be formed of Li, Mg, Al, Al—Li, Ca, Mg—In, Mg—Ag, or the like. Alternatively, a transparent cathode formed of, for example, ITO or IZO can be used to produce a front surface light emitting diode.

The method of manufacturing an organic light emitting diode, according to an exemplary embodiment of the present invention, includes: forming a first electrode on the substrate; forming an organic layer comprising the aromatic heterocyclic compound of Formula 1 on the first electrode; and forming a second electrode on the organic layer. Herein, the organic layer may be an EML, a HIL, a HTL, a HBL, or ETL. In addition, the manufacturing method of the organic light emitting diode may further include, if necessary, forming at least one layer selected from the group consisting of, for example, the HIL, the HTL, the EML, the HBL, the ETL, and an EIL.

The forming of the organic layer comprising the aromatic heterocyclic compound of Formula 1 may be performed by, for example, vacuum deposition or solution coating such as spin coating, inkjet printing, screen printing, casting, LB, or spray printing. In addition, the organic layer comprising the aromatic heterocyclic compound of Formula 1 may be formed, for example, on a donor film using vacuum deposition or solution coating as described above, and then thermally transferred to a substrate on which a first electrode and the like are formed.

Hereinafter, Synthesis Examples and Examples of the present invention will be described in detail. However, these examples are provided to facilitate the understanding of the present invention only, and are not intended to limit the scope of the present invention.

### EXAMPLE

### Synthesis Example 1

Compound 1 was synthesized through Reaction Scheme 1 below:

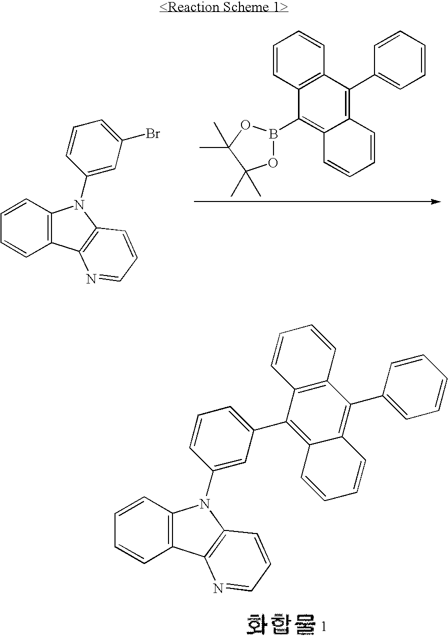

about 3 g of 5-(3-bromophenyl)-5H-pyrido[3,2-b]indole (about 9.28 mmol), about 4.24 g of 2-(9-phenylanthracen-10-yl)-4,4,5,5-tetramethyl-[1,3,2]-dioxaborolane) (about 11.14 mmol), and about 0.54 g of tetrakis(triphenylphosphine)palladium[0] (about 0.464 mmol) were dissolved in about 60 ml of THF, and about 20 ml of an aqueous sodium carbonate solution (about 11.14 mmol) was added to the mixture. Then, the mixture was refluxed for about 24 hours. After reflux, the resultant was extracted using methylene chloride, and a column chromatography was performed on the resultant to obtain about 3.9 g of 5-(3-(10-phenylanthracen-9-yl)phenyl)-5H-pyrido[3,2-b]indole (yield about 84.6%).

¹H NMR (about 300 MHz, CDCl₃) δ about 7.30-about 7.89 (m, about 22H), about 8.47 (d, J=about 7.6 Hz, about 1H), about 8.62 (d, J =about 4.7 Hz, about 1H)

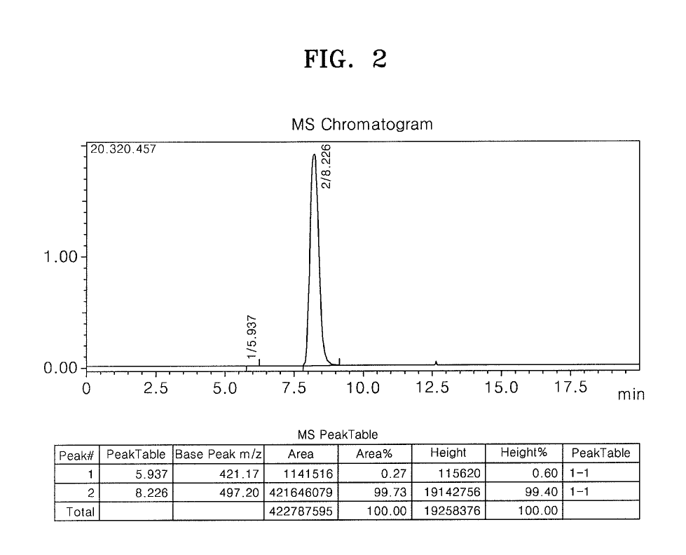

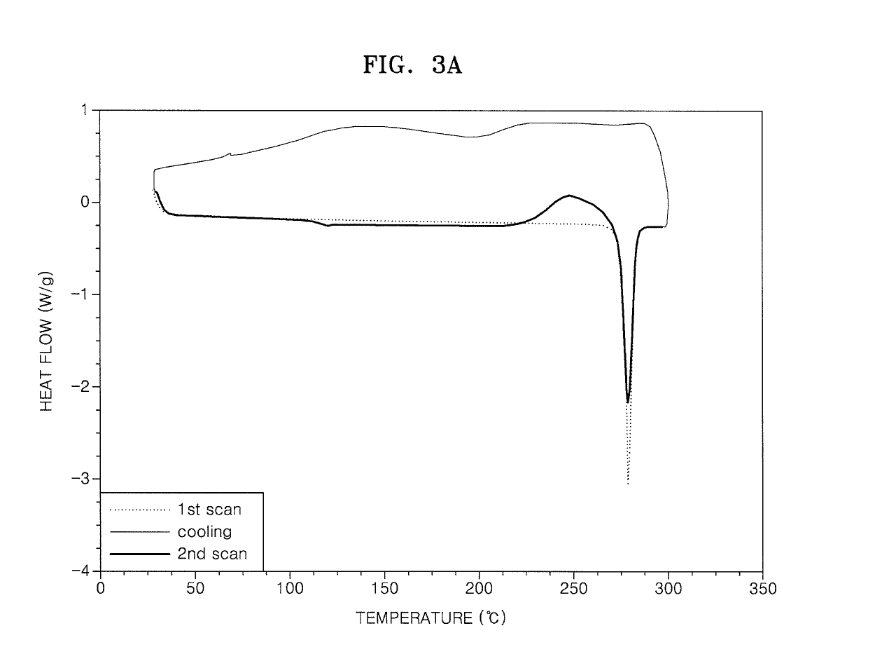

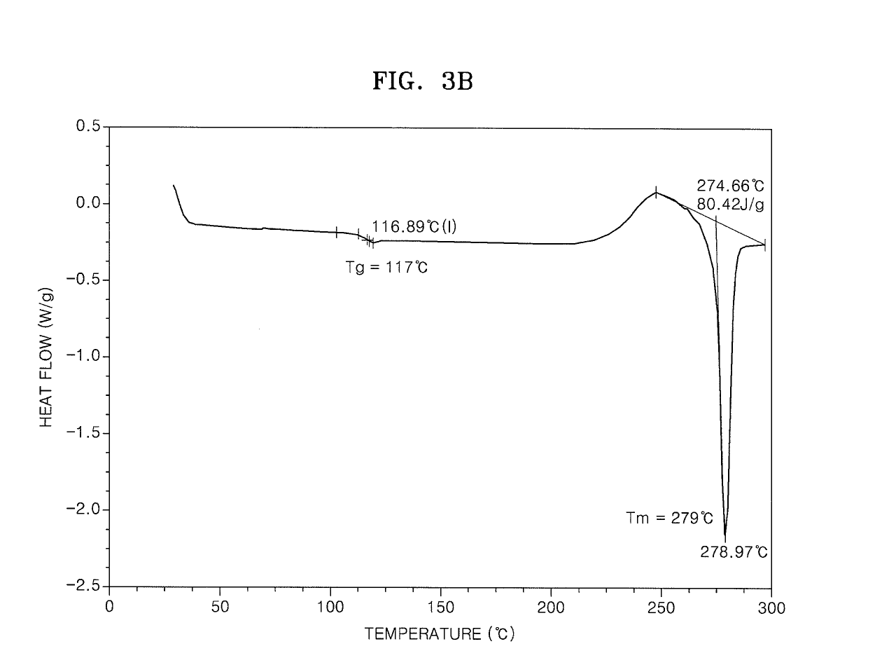

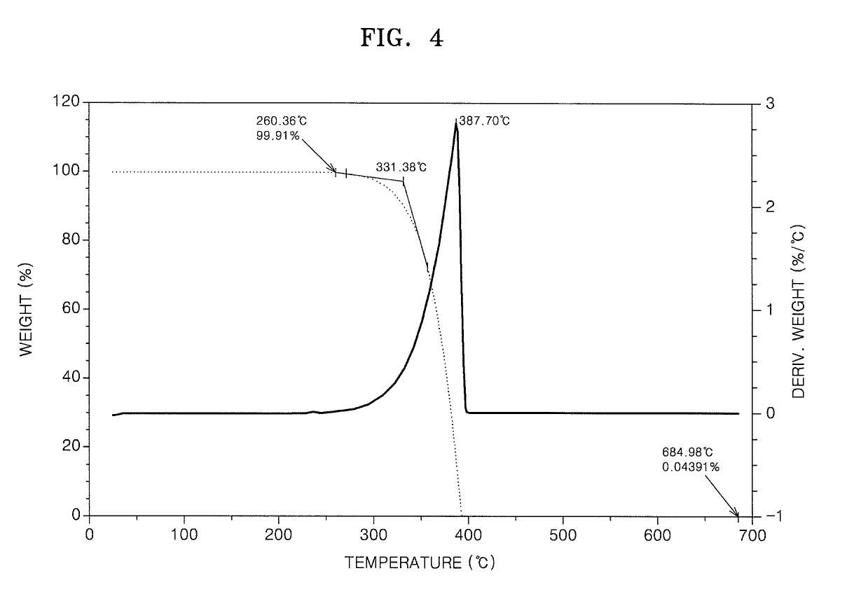

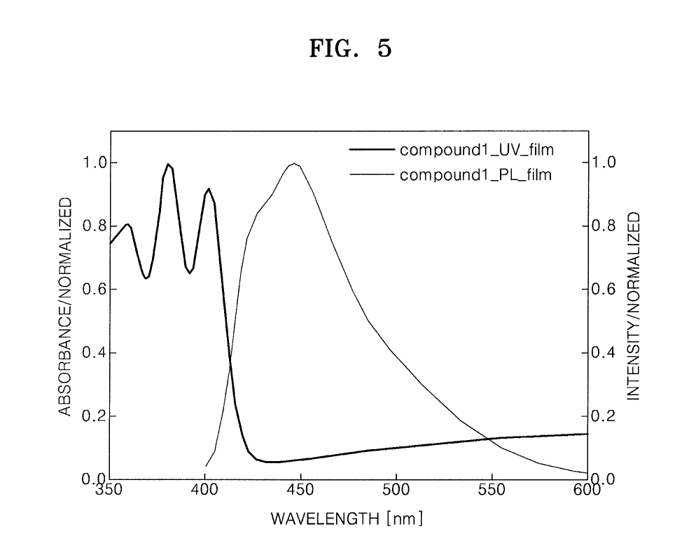

Thermal analysis of Compound 1 was performed using thermo gravimetric analysis (TGA) and differential scanning calorimetry (DSC) (N₂ atmosphere, temperature range: room temperature to about 600° C. (about 10° C./min)-TGA, room temperature to about 400° C.-DSC, Pan Type: Pt Pan in disposable Al Pan (TGA), disposable Al pan (DSC)). As a result, it was confirmed that Compound 1 had a Tg of about 117° C., a Tm of about 279° C., and a Td of about 331° C. FIG. 2 is a graph showing MS chromatography results of Compound 1, FIGS. 3A and 3B are graphs showing DSC data of Compound 1, FIG. 4 is a graph showing TGA data of Compound 1, and FIG. 5 is a graph showing UV and PL data of a film formed of Compound 1.

Also, the HOMO of Compound 1 had an energy of about −5.249 eV, the LUMO of Compound 1 had an energy of about −1.767 eV, and an energy gap between the HOMO and LUMO was about 3.482 eV (the values described above are calculated values). On the other hand, the HOMO of Compound 1 had an energy of about −5.89 eV, the LUMO of Compound 1 had an energy of about −2.90 eV, and an energy gap between the HOMO and LUMO was about 2.99 eV (the values described above are actually measured values from AC2 and UV edge).

### Synthesis Example 2

Compound 6 was synthesized through Reaction Scheme 2 below:

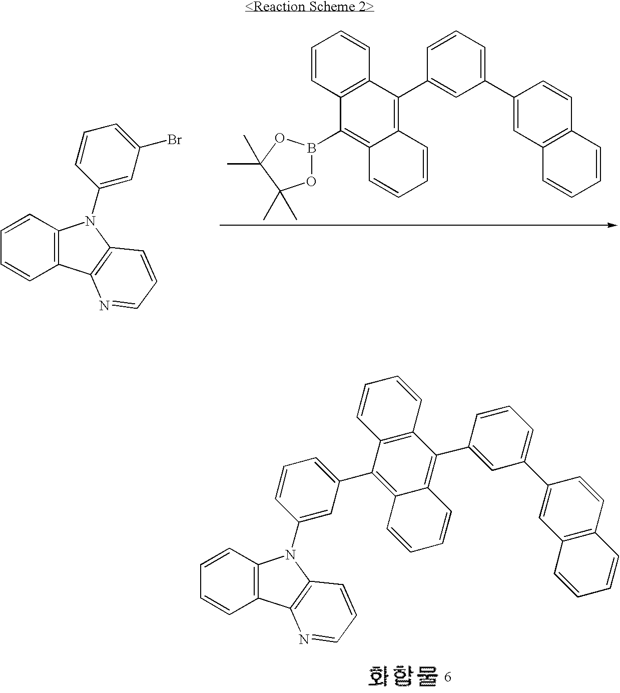

about 1.6 g of 5-(3-bromophenyl)-5H-pyrido[3,2-b]indole (about 4.93 mmol), about 3 g of 4,4,5,5-tetramethyl-2-(10-(3-(naphthalen-2-yl) phenyl)anthracen-9-yl)-1,3,2-dioxaborolane (about 5.92 mmol), and about 0.29 g of tetrakis(triphenylphosphine)palladium[0] were dissolved in about 50 ml of THF, and about 15 ml of an aqueous sodium carbonate solution (about 5.92 mmol) was added to the mixture. Then, the mixture was refluxed for about 24 hours. After reflux, the resultant was extracted using methylene chloride, and a column chromatography was performed on the resultant to obtain about 2.7 g of 5-(3-(10-(3-(naphthalen-2-yl)phenyl)anthracen-9-yl)phenyl)-5H-pyrido[3,2-b]indole (yield about 88%).

¹H NMR (about 300 MHz, CDCl₃) δ about 7.38-about 7.96 (m, about 28H), about 8.14 (d, J=about 9.4 Hz, about 1H), about 8.63 (d, J=about 4.8 Hz, about 1H)

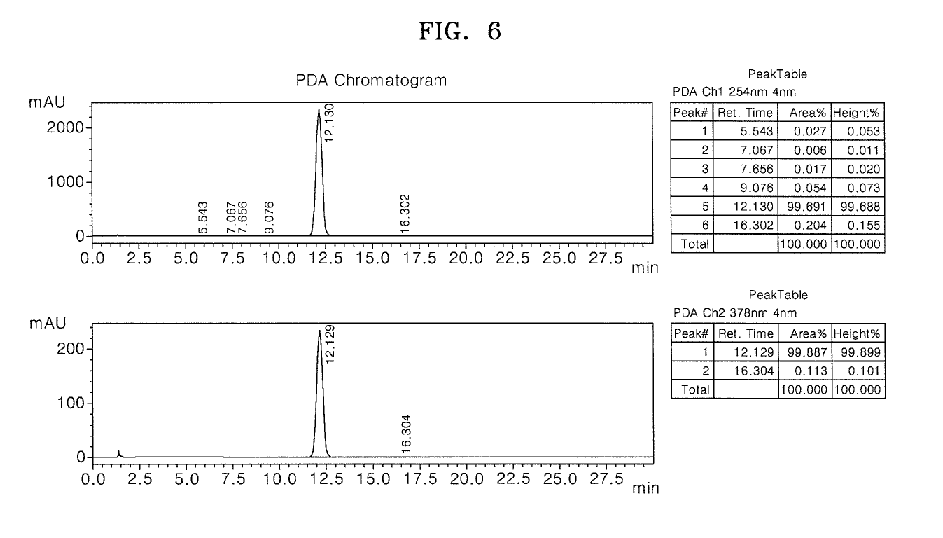

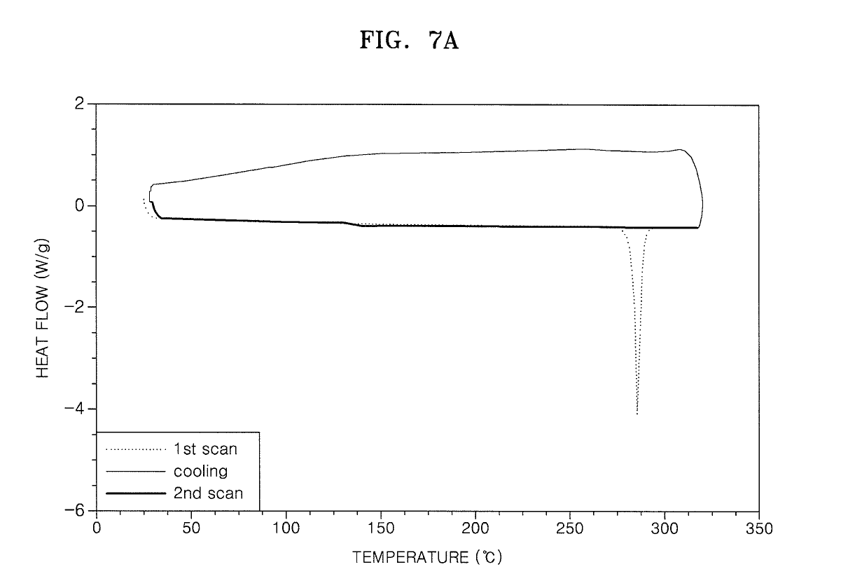

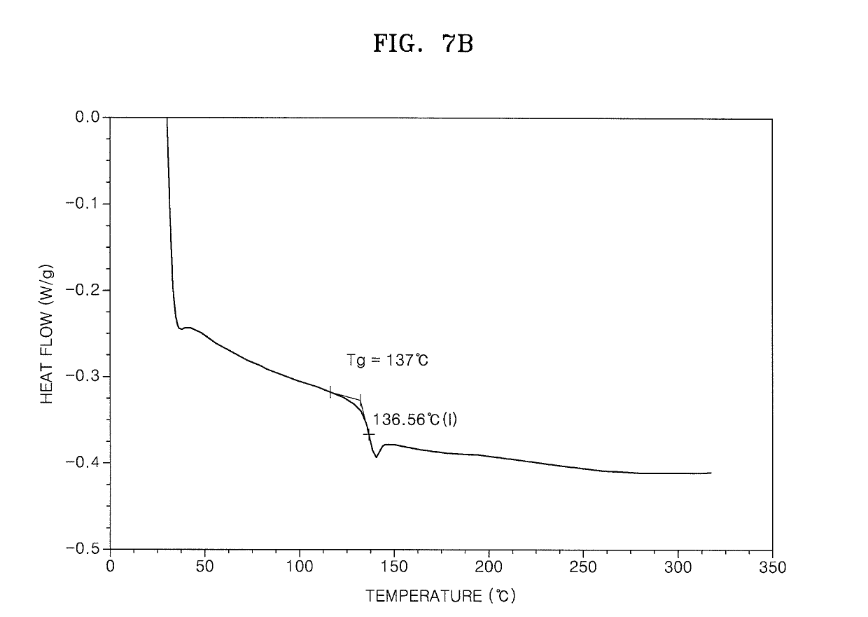

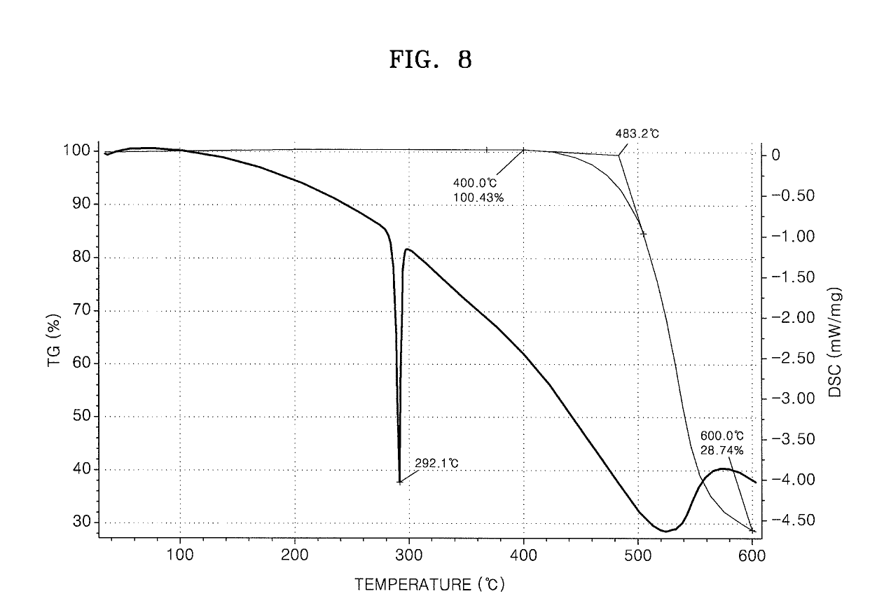

Thermal analysis of Compound 6 was performed using TGA and DSC (N₂ atmosphere, temperature range: room temperature to about 600° C. (about 10° C./min)-TGA, room temperature to about 400° C.-DSC, Pan Type: Pt Pan in disposable Al Pan (TGA), disposable Al pan (DSC)). As a result, it was confirmed that Compound 6 had a Tg of about 137° C., a Tm of about 286° C., and a Td of about 483° C. FIG. 6 is a graph showing MS chromatography results of Compound 6, FIGS. 7A and 7B are graphs showing DSC data of Compound 6, and FIG. 8 is a graph showing TGA data of Compound 6.

In addition, the LUMO of Compound 6 had an energy of about −1.774 eV, the HOMO of Compound 6 had an energy of about −5.250 eV, and an energy gap between the LUMO and HOMO was about 3.475 eV (the values described above are calculated values).

### Synthesis Example 3

Compound 7 was synthesized through Reaction Scheme 3 below:

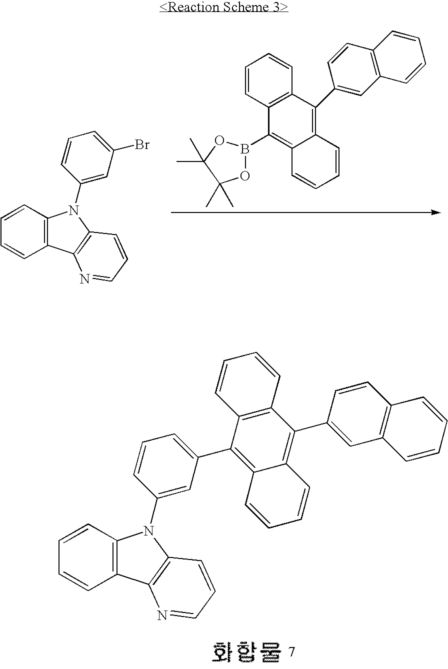

about 4.73 g of 5-(3-bromophenyl)-5H-pyrido[3,2-b]indole (about 14.63 mmol), about 7.56 g of 4,4,5,5-tetramethyl-2-(10-(3-(naphthalen-2-yl)phenyl)anthracen-9-yl)-1,3,2-dioxaborolane (about 17.56 mmol), and about 0.84 g of tetrakis(triphenylphosphine)palladium[0] (0.73mmol) were dissolved in about 90 ml of THF, and about 30 ml of an aqueous sodium carbonate solution (about 17.56 mmol) was added to the mixture. Then, the mixture was refluxed for about 24 hours. After reflux, the resultant was extracted using methylene chloride, and a column chromatography was performed on the resultant to obtain about 5.6 g of 5-(3-(10-(naphthalen-2-yl)anthracen-9-yl)phenyl)-5H-pyrido[3,2-b]indole (yield about 70.0%).

¹H NMR (about 300 MHz, CDCl₃) δ about 7.26 to about 7.47 (m, about 6H), about 7.58 to about 8.11 (m, about 19H), about 8.6 (d, J=about 11.2 Hz, about 1H)

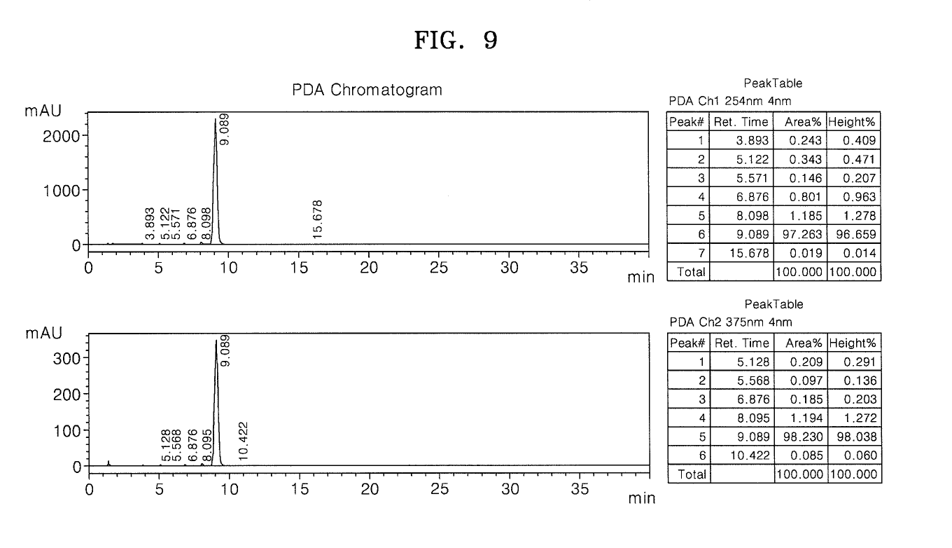

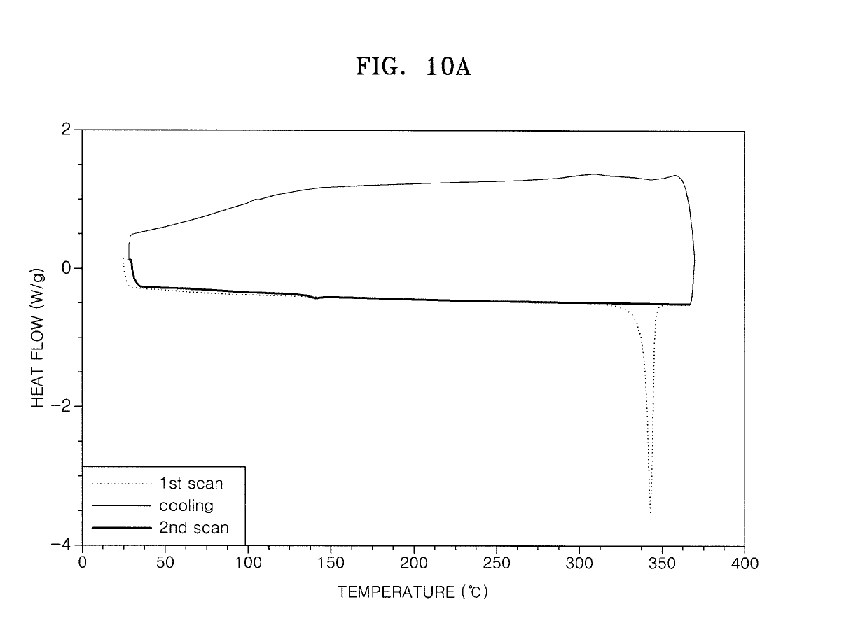

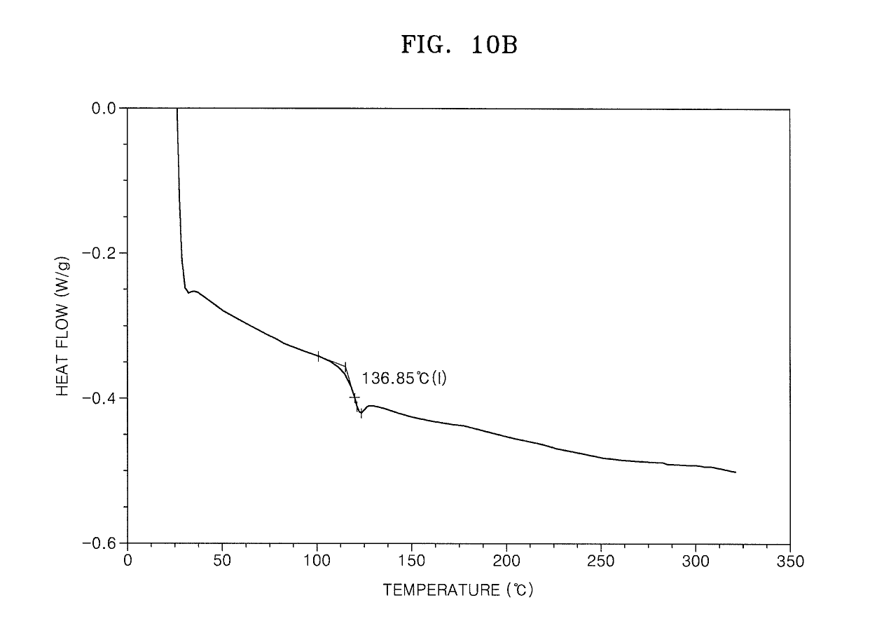

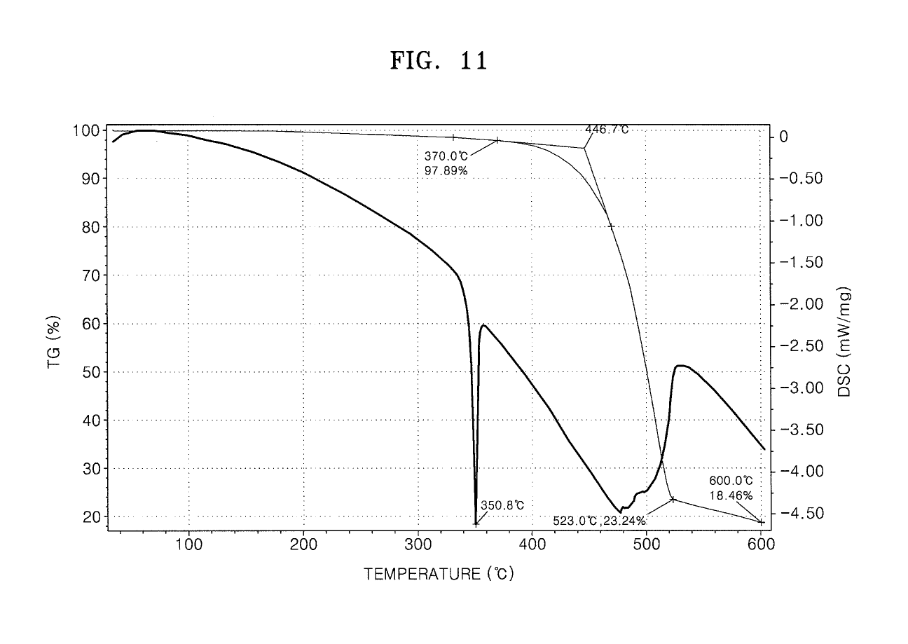

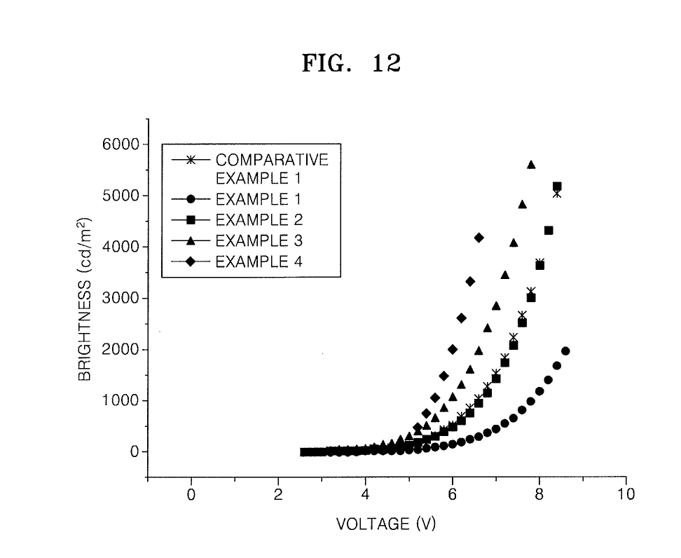

Thermal analysis of Compound 7 was performed using TGA and DSC (N₂ atmosphere, temperature range: room temperature to 600° C. (10° C./min)-TGA, room temperature to 400° C.-DSC, Pan Type: Pt Pan in disposable Al Pan (TGA), disposable Al pan (DSC)). As a result, it was confirmed that Compound 7 had a Tg of 139° C., a Tm of 344° C., and a Td of 447° C. FIG. 9 is a graph showing MS chromatography results of Compound 7, FIGS. 10A and 10B are graphs showing DSC data of Compound 7, and FIG. 11 is a graph showing TGA data of Compound 7.

In addition, the LUMO of Compound 7 had an energy of about −1.764 eV, the HOMO of Compound 7 had an energy of about −5.274 eV, and an energy gap between the LUMO and HOMO was about 3.511 eV (the values described above are calculated values).

### Evaluation Example 1

### Evaluation of Light Emitting Properties of Compounds (Solution State)

Evaluation of light emitting properties of Compound 1 was performed by evaluating UV absorption and photoluminescence (PL) spectra of Compound 1.

Compound 1 was diluted in toluene to the concentration of about 0.2 mM, and then the UV absorption spectrum thereof was measured using a Shimadzu UV-350 Spectrometer. In addition, Compound 1 was diluted in toluene to the concentration of about 10 mM, and the PL spectrum thereof was measured using an ISC PC1 spectrofluorometer equipped with a Xenon lamp. As a result, it was confirmed that the maximum absorption wavelength of the UV absorption spectrum of Compound 1 was about 400 nm, and the maximum wavelength of the PL spectrum of Compound 1 was about 445 nm.

### Example 1

By using Compound 1 as a material used to form an emissive layer (EML), an organic light emitting diode having the following structure was manufactured: ITO//HIL(about 60 nm)//HTL(about 30 nm)//EML(about 35 nm)//ETL(about 25 nm)//LiF(about 0.6 nm)/Al(about 150 nm).

As an anode, an about 15 ΩD/cm² (about 1000 Å) ITO glass substrate was cut to a size of about 50 mm×about 50 mm×about 0.7 mm, ultrasonic washed with acetone isopropyl alcohol and pure water for about 15 minutes each, and then washed with UV ozone for about 30 minutes. MTDATA as a material used to form a hole injection layer (HI L) was vacuum deposited on the ITO anode at a deposition speed of about 1 Å/sec to form the HIL having a thickness of about 60 nm, and then α-NPD was vacuum deposited on the HIL at a deposition speed of about 1 Å/sec to form a hole transport layer (HTL) having a thickness of about 30 nm. Then, Compound 1 was vacuum deposited on the HTL at a deposition speed of about 1 Å/sec to form an emissive layer (EML) having a thickness of about 35 nm. Subsequently, Alq₃ was vacuum deposited on the EML to form an electron transport layer (ETL) having a thickness of about 25 nm. Then, LiF was vacuum deposited on the ETL to form an electron injection layer (EIL) having a thickness of about 0.6 nm, and Al was vacuum deposited on the EIL to form a cathode having a thickness of about 150 nm to complete the manufacture of an organic light emitting diode as illustrated in FIG. 1B. This is referred to as Sample 1.

### Example 2

An organic light emitting diode was manufactured in the same manner as in Example 1, except that the EML was formed using Compound 1 as a host and DPAVBi as a dopant (doping concentration of about 5 wt %), instead of using only Compound 1 as a material used to form the EML. This is referred to as Sample 2. Herein, Compound 1 (host) and DPAVBi (dopant) were respectively deposited at a deposition speed of about 1 Å/sec and at a deposition speed of about 0.05 Å/sec.

### Example 3

An organic light emitting diode was manufactured in the same manner as in Example 2, except that the HTL was formed to a thickness of about 20 nm. This is referred to as Sample 3.

### Example 4

An organic light emitting diode was manufactured in the same manner as in Example 2, except that the EML was formed of ADN (9, 10-dinaphthylanthracene) as a host and DPAVBi (doping concentration of about 5 wt %) as a dopant, instead of using Compound 1 as a host and DPAVBi as a dopant (doping concentration of about 5 wt %), and the ETL was formed of Compound 1, instead of using Alq₃. This is referred to as Sample 4.

### Comparative Example 1

An organic light emitting diode was manufactured in the same manner as in Example 2, except that the EML was formed of ADN (9, 10-dinaphthylanthracene) as a host and DPAVBi (doping concentration of about 5 wt %) as a dopant, instead of using Compound 1 as a host and DPAVBi as a dopant (doping concentration of about 5 wt %). This is referred to as Sample A.

### Evaluation Example 2

Turn-on voltage, power, luminance and color coordinate of each of Samples 1 through 4 and A were evaluated using a PR650 (Spectroscan) Source Measurement Unit. The results are shown in Table 2 below.

TABLE 2

| | Turn | | | |
|---|---|---|---|---|
| | on/Op. | Im/W@1000nit | Cd/A@1000nit | CIE |
| | V@1000nit | (max@V) | (max@V) | @1000nit |
| Comparative | about 3.4 V/ | about 3.36 | about 7.06 | about |
| Example 1 | about 6.6 V | (about 4.73@ | (about 7.15@ | 0.14, |
| | | about 4.2 V) | about 7.4 V) | about |
| | | | | 0.26 |
| Example 1 | about 3.4 V/ | about 0.32 | about 0.80 | about |
| | about 7.8 V | (about 0.43@ | (about 0.80@ | 0.17, |
| | | about 4.4 V) | about 7.6 V) | about |
| | | | | 0.29 |
| Example 2 | about 3.0 V/ | about 1.11 | about 2.34 | about |
| | about 6.6 V | (about 1.52@ | (about 2.40@ | 0.15, |
| | | about 3.6 V) | about 7.4 V) | about |
| | | | | 0.26 |
| Example 3 | about | about 1.21 | about 2.32 | about |
| | 2.8 V/about | (about | (about 2.37@ | 0.15, |
| | 6.0 V | 2.14@about | about 6.8 V) | about |
| | | 3.0 V) | | 0.28 |
| Example 4 | about 3.4 V/ | about 2.99 | about 5.33 | about |
| | about 5.6 V | (about | (about 5.55@ | 0.14, |
| | | 3.22@about | about 6.2 V) | about |
| | | 4.0 V) | | 0.26 |

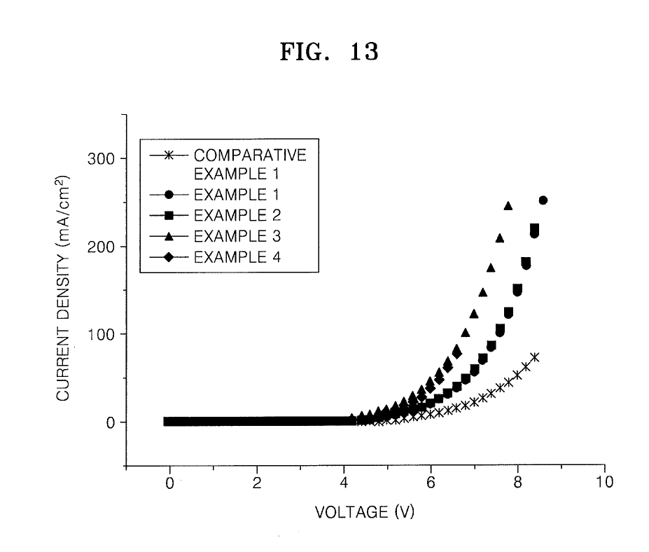

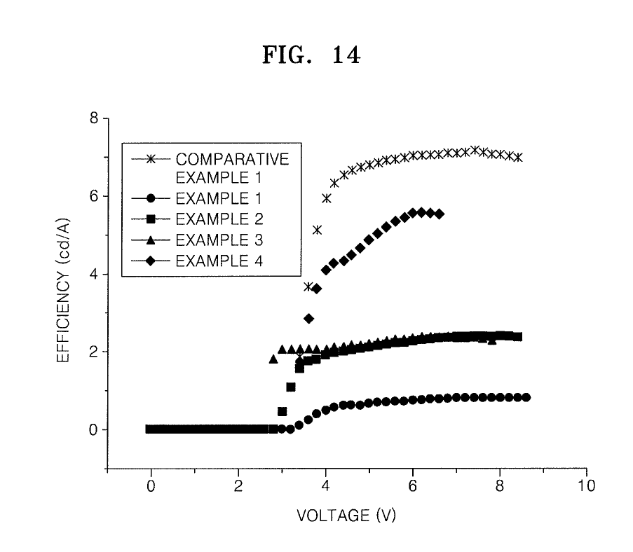

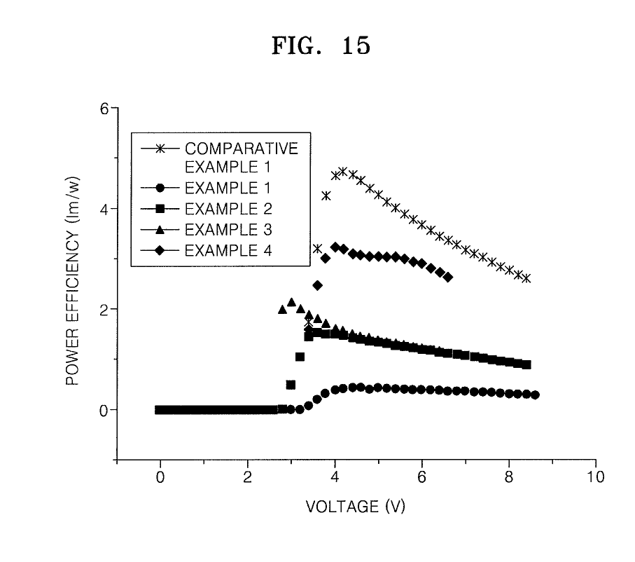

In addition, graphs (brightness, current density, efficiency and power efficiency) showing the evaluation results of Samples 1 through 4 and A (Examples 1 through 4 and Comparative Example 1) are illustrated in FIGS. 12, 13, 14, and 15.

Having described the exemplary embodiments of the present invention, it is further noted that it is readily apparent to those of reasonable skill in the art that various modifications may be made without departing from the spirit and scope of the invention which is defined by the metes and bounds of the appended claims.

What is claimed is:

1. An aromatic heterocyclic compound represented by Formula 1 below: 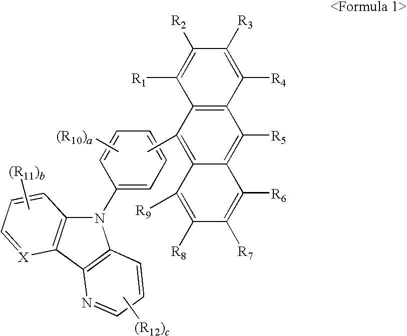
wherein R₁ through R₉ are each independently hydrogen, halogen, a cyano group, a nitro group, a hydroxyl group, a substituted or unsubstituted C₁-C₂₀ alkyl group, a substituted or unsubstituted C₂-C₂₀ alkenyl group, a substituted or unsubstituted C₂-C₂₀ alkynyl group, a substituted or unsubstituted C₅-C₂₀ cycloalkyl group, a substituted or unsubstituted C₅-C₂₀ cycloalkenyl group, a substituted or unsubstituted C₅-C₂₀ aryl group, a substituted or unsubstituted C₂-C₃₀ heteroaryl group, or a group represented by —N(Z₁)(Z₂) where Z₁ and Z₂ are each independently hydrogen, a substituted or unsubstituted C₁-C₂₀ alkyl group, or a substituted or unsubstituted C₅-C₂₀ aryl group,
R₁₀ through R₁₂ are each independently hydrogen, halogen, a cyano group, a nitro group, a hydroxyl group, a substituted or unsubstituted C₁-C₂₀ alkyl group, a substituted or unsubstituted C₂-C₂₀ alkenyl group, a substituted or unsubstituted C₂-C₂₀ alkynyl group, a substituted or unsubstituted C₅-C₂₀ cycloalkyl group, a substituted or unsubstituted C₅-C₂₀ cycloalkenyl group, a substituted or unsubstituted C5-C₂₀ aryl group, a substituted or unsubstituted C₂-C₂₀ heteroaryl group, a group represented by —N(Z₁)(Z₂) where Z₁ and Z₂ are each independently hydrogen, a substituted or unsubstituted C₁-C₂₀ alkyl group, or a substituted or unsubstituted C₅-C₂₀ aryl group, or a group represented by -Q₁-Q₂ where Q₁ is a C₅-C₂₀ arylene group or a C₂-C₂₀ heteroarylene group, and Q₂ is a substituted or unsubstituted C₅-C₂₀ aryl group or a substituted or unsubstituted C₂-C₂₀ heteroaryl group,
a, b, and c are each independently 0, 1 or 2; and
X is CY₁ or N, wherein Y₁ is hydrogen, a substituted or unsubstituted C₁-C₂₀ alkyl group, or a substituted or unsubstituted C₁-C₂₀ alkoxy group.

2. The aromatic heterocyclic compound of claim 1, wherein the compound is represented by Formula 1A below:

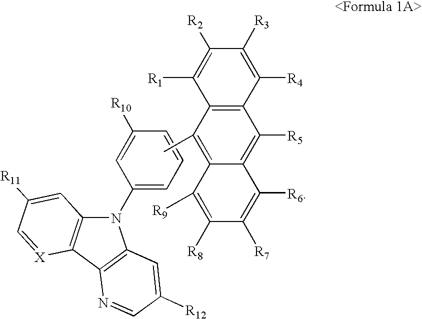

3. The aromatic heterocyclic compound of claim 1, wherein R₁₀ through R₁₂ are each independently hydrogen, a substituted or unsubstituted C₁-C₂₀alkyl group, a substituted or unsubstituted C₅-C₂₀aryl group, a substituted or unsubstituted C₂-C₂₀ heteroaryl group, or a group represented by -Q₁-Q₂ where Q₁ is a C₅-C₂₀ arylene group, and Q₂ is a substituted or unsubstituted C₅-C₂₀ aryl group or a substituted or unsubstituted C₂-C₂₀ heteroaryl group.

4. The aromatic heterocyclic compound of claim 1, wherein R₁₀ through R₁₂ are each independently hydrogen or one of the structures represented by Formula 2a to 2o below:

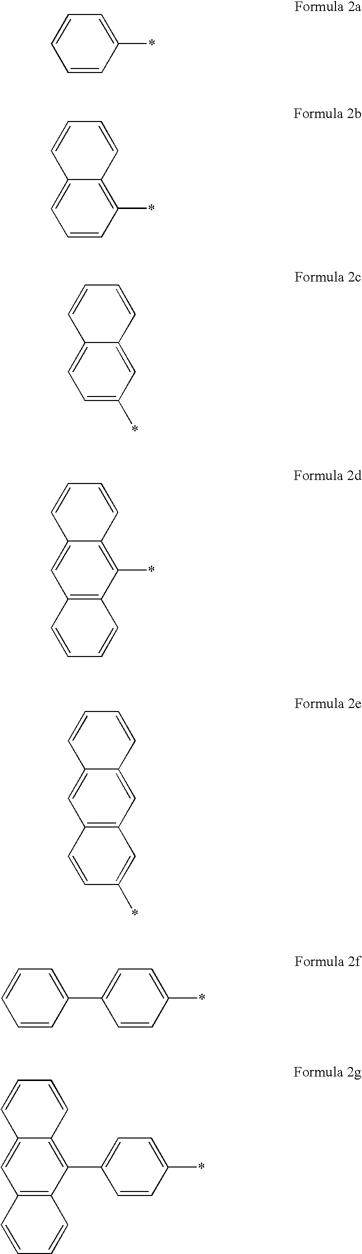

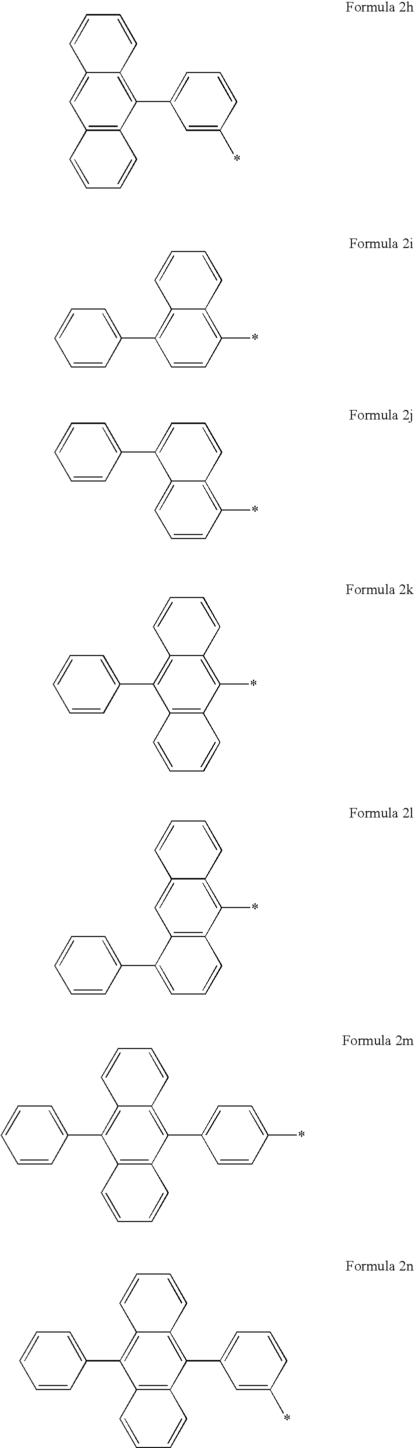

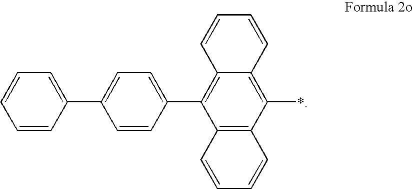

5. The aromatic heterocyclic compound of claim 1, wherein X is CH or N.

6. The aromatic heterocyclic compound of claim 1, wherein R₁ through R₉ are each independently hydrogen, a substituted or unsubstituted C₅-C₂₀ aryl group or a substituted or unsubstituted C₂-C₃₀ heteroaryl group.

7. The aromatic heterocyclic compound of claim 1, wherein R₁ through R₄ and R₆ through R₉ are hydrogen, and R₅ is hydrogen, a substituted or unsubstituted C₅-C₂₀ aryl group or a substituted or unsubstituted C₂-C₃₀ heteroaryl group.

8. The aromatic heterocyclic compound of claim 1, wherein R₁ through R₉ are each independently hydrogen or one of the structures represented by Formula 3a to 3i below:

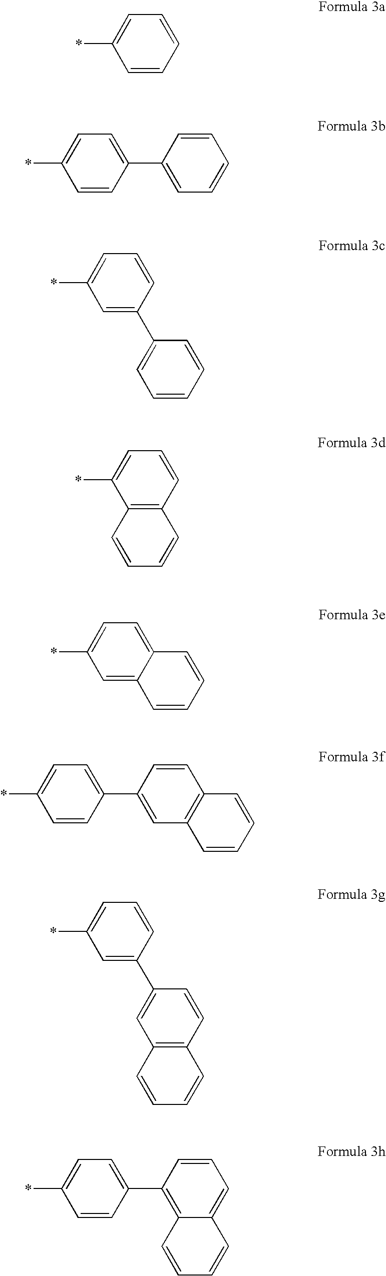

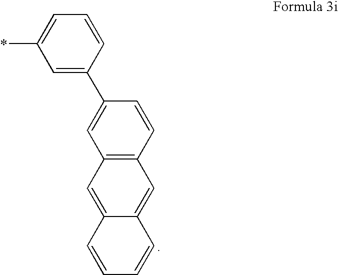

9. The aromatic heterocyclic compound of claim 1, wherein the compound is represented by Formula 1B below: 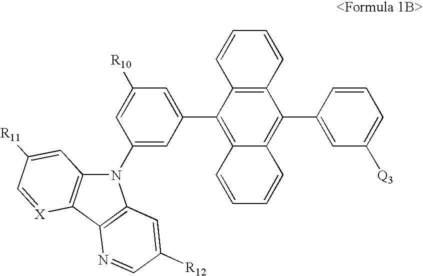
wherein R₁₀ through R₁₂ are each independently hydrogen, halogen, a cyano group, a nitro group, a hydroxyl group, a substituted or unsubstituted C₁-C₂₀ alkyl group, a substituted or unsubstituted C₂-C₂₀ alkenyl group, a substituted or unsubstituted C₂-C₂₀ alkynyl group, a substituted or unsubstituted C₅-C₂₀ cycloalkyl group, a substituted or unsubstituted C₅-C₂₀ cycloalkenyl group, a substituted or unsubstituted C₅-C₂₀ aryl group, a substituted or unsubstituted C₂-C₂₀ heteroaryl group, a group represented by —N(Z₁)(Z₂) where Z₁ and Z₂ are each independently hydrogen, a substituted or unsubstituted C₁-C₂₀ alkyl group, or a substituted or unsubstituted C₅-C₂₀ aryl group, or a group represented by -Q₁-Q₂ where Q₁ is a C₅-C₂₀ arylene group or a C₂-C₂₀ heteroarylene group, and Q₂ is a substituted or unsubstituted C₅-C₂₀ aryl group or a substituted or unsubstituted C₂-C₂₀ heteroaryl group;
X is CH or N; and
Q₃ is hydrogen or an unsubstituted C₅-C₂₀ aryl group.

10. The aromatic heterocyclic compound of claim 1, wherein the compound is one of Compounds 1 through 17 below:

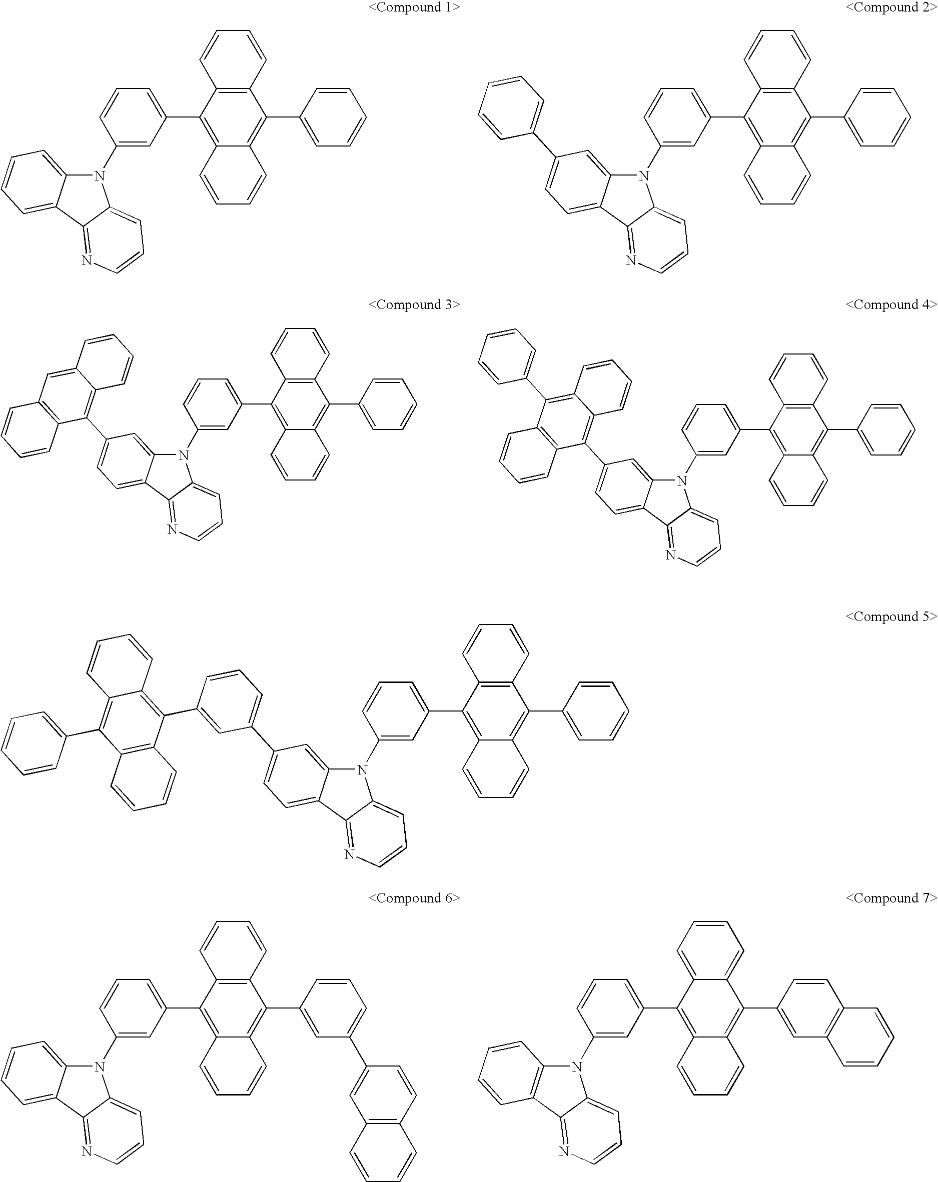

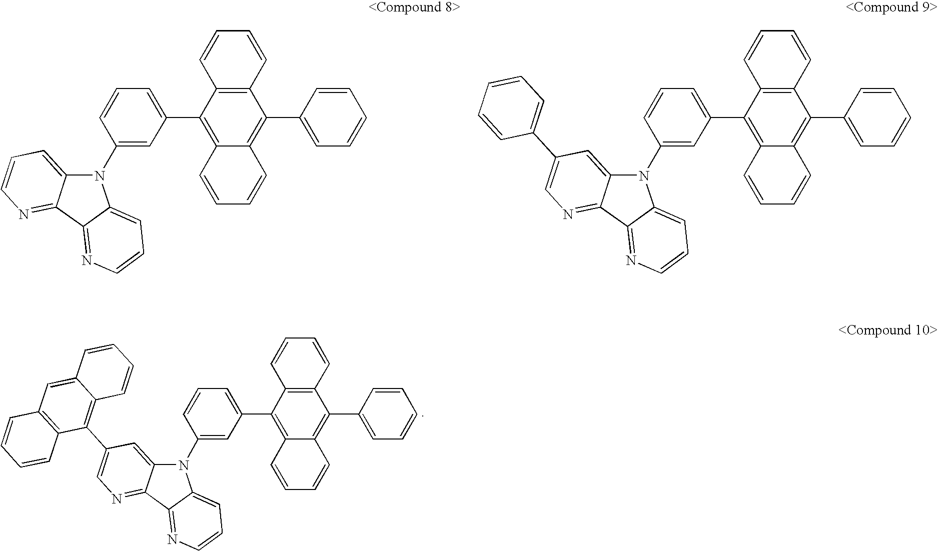

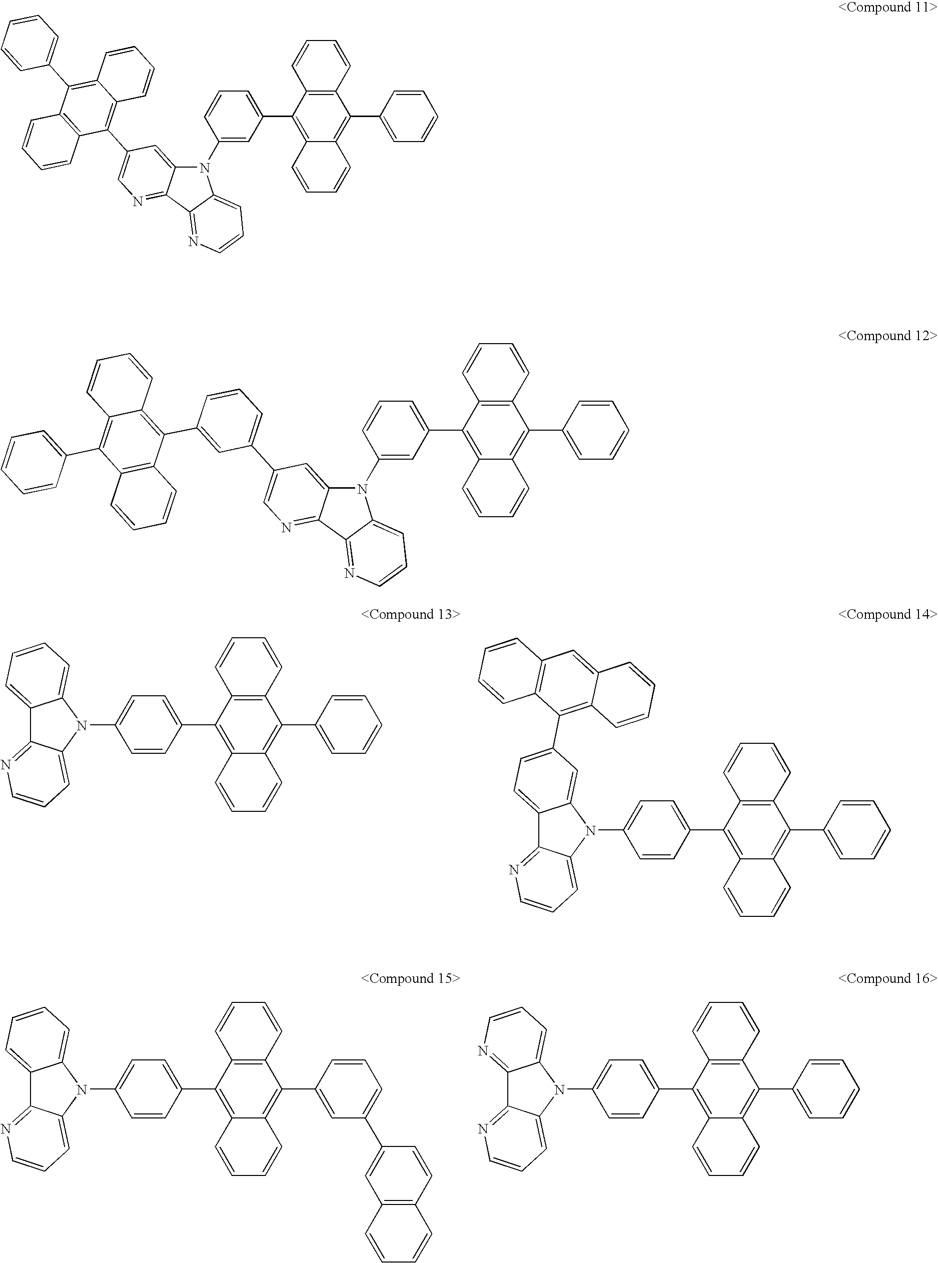

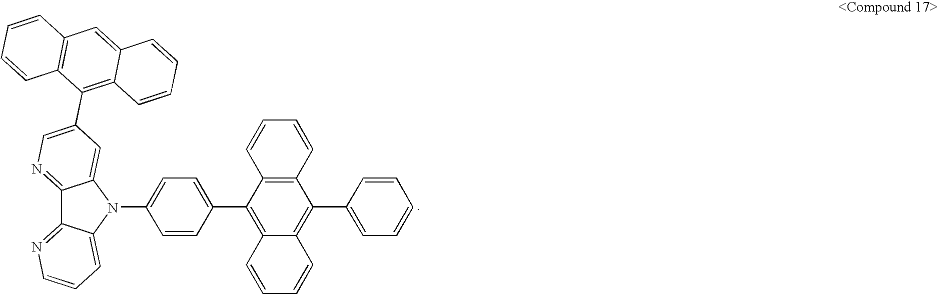

11. An organic light emitting diode comprising a first electrode, a second electrode, and an organic layer comprising the aromatic heterocyclic compound according to claim 1 located between the first electrode and the second electrode.

12. The organic light emitting diode of claim 11, wherein the organic layer is an emissive layer or an electron transport layer.

13. The organic light emitting diode of claim 11, further comprising, at least one additional layer selected from the group consisting of a hole injection layer, a hole transport layer, an emissive layer, a hole blocking layer, an electron transport layer, and an electron injection layer located between the first electrode and the second electrode.

14. A method of manufacturing an organic light emitting diode, the method comprising:
forming a first electrode on a substrate;
forming an organic layer comprising the aromatic heterocyclic compound according to claim 1 on the first electrode; and
forming a second electrode on the organic layer.

15. The method of claim 14, wherein the forming of the organic layer is performed using one of vacuum deposition, spin coating, inkjet printing, screen printing, casting, Langmuir Blodgett (LB) deposition, spray printing, or thermal transfer.
